(19) United States
(12) Patent Application Publication
(10) Pub. No.: US 2026/0227397 A1
(43) Pub. Date: Aug. 6, 2026

(54) METHODS AND SYSTEMS OF PROFILING WITH BIOLOGICAL COMPUTING CELLS

(71) Applicants: Daniel GEORGIEV, Pilsen (CZ); XENO CELL INNOVATIONS S.R.O., Pilsen (CZ)

(72) Inventors: Daniel GEORGIEV, Pilsen (CZ); Martin CIENCIALA, Pilsen (CZ); Hynek KASL, Pilsen (CZ); Laura BERNE, Pilsen (CZ)

(21) Appl. No.: 19/148,914

(22) PCT Filed: Jan. 17, 2024
(86) PCT No.: PCT/US2024/011802

§ 371 Date: Jul. 17, 2025

Related U.S. Application Data
(60) Provisional application No. 63/480,457, filed on Jan. 18, 2023.

(51) Int. Cl. G01N 33/569; G01N 21/64; G01N 33/58

(52) U.S. Cl. CPC G01N 33/56966; G01N 21/6428; G01N 33/582; G01N 2021/6439; G01N 2333/71; G01N 2510/00

(57) ABSTRACT

Provided herein are biological computing methods, systems, and kits, which can include computing units that can process input signals to produce output signals. Computing units can include cells and/or molecules that can convert biological signals into output signals that can provide information about the input sample.

### CROSS-REFERENCE TO RELATED APPLICATIONS

This application claims the benefit of priority to U.S. Provisional Patent Application Ser. No. 63/480,457, filed on Jan. 18, 2023, which is incorporated herein by reference in its entirety for all purpose.

### INCORPORATION OF THE SEQUENCE LISTING

This application contains a Sequence Listing. The material in the accompanying Sequence Listing is hereby incorporated by reference into this application. The accompanying Sequence Listing XML file, named “2024-01-12 Sequence_Listing_054357-503001WO_ST26.xml,” was created on Jan. 12, 2024, and is 11,251 bytes.

### BACKGROUND

To gain a comprehensive understanding of cells, cells need to be studied on both multi- and single-cell levels. However, heterogeneity within the smallest populations of cells is a natural property of multi-cellular organisms and presents major challenges in drug and biomarker discoveries and developments. Thus, the emerging field of single cell research is facing a limitation of processing power. Analyzing each single cells in a population requires processing hundreds of thousands to millions of single cells. Currently existing techniques, such as molecule-based enzymatic methods (e.g., enzyme-linked immunosorbent assay (ELISA)) and instrument-based brute force methods (e.g., flow cytometry), are limited in, inter alia, its processing power. For example, analyzing a group of cells in a sample size of a human body either by ELISA or flow cytometry would take a half a year. The emerging field of single cell research is facing a bottleneck between discovery of new drugs and biomarkers and their translation into accessible assays and diagnostics. Therefore, there is a need for a new single-cell technology that can analyze as many cells in a short amount of time, not years.

### SUMMARY

The present disclosure provides an innovative platform (biocytometry) that allows analyzing as many cells in a day as the other technologies, e.g., flow cytometry, can currently do in a year, with unprecedented levels of sensitivity and specificity. Several aspects described herein relate to biological computing systems and kits, and methods of using such systems and kit.

Accordingly, in one aspect, provided herein is a method of detecting presence or absence of an output signal indicative of a characteristic of an input sample, which can include: (a) contacting at least one sample object derived from the input sample with a Reaction Reagent including at least one computing unit (CU) and with a Reaction Medium, wherein the at least one CU can be configured to interact with the at least one sample object; (b) incubating (a) for a sufficient amount of time in an incubator for the output signal to be generated; (c) adding a readout reagent including a reporter entity to (b), wherein the reporter entity can generate the output signal; and (d) measuring the output signal, wherein the output signal can be directly proportional to the amount of the at least one sample object derived from the input sample, thereby detecting the presence or absence of the output signal indicative of the characteristic of the input sample. In some embodiments, the contacting in step (a) can include first (i) adding the at least one sample objects to the Reaction Reagent and then (ii) adding the Reaction Medium to (i). In some embodiments, the sufficient amount of time in step (b) can be about 4 hours.

In some embodiments, in any of the methods described herein, the characteristic of the input sample can be apoptosis, activation, suppression, anergy, or differentiation.

In some embodiments, in any of the methods described herein, the output signal may not include any background signal regardless of the input sample type.

In some embodiments, in any of the methods described herein, the input sample can be, inter alia, whole blood, a stabilized leukocyte fraction, isolated PBMCs, cryogenically stored PBMCs, primary cell cultures, cell lines, tumor samples, stem cells, or CAR-T cells.

In some embodiments, the Reaction Reagent can contain the at least one CU and hydrogel. In some embodiments, the hydrogel can be gelatin. In some embodiments, the Reaction Reagent can further contain yeast extract, peptone, D-glucose, Dulbecco's phosphate-buffered saline, D-(+)-Trehalose dihydrate, and skim milk. In some embodiments, the Reaction Reagent can be stored up to about 2 weeks at room temperature without loss of signal. In some embodiments, the Reaction Reagent can be stored up to about 8 weeks at 4° C. without loss of signal.

In some embodiments, the Reaction Medium can be a semi-permeable nutritive medium with thermoresponsive properties. In some embodiments, the Reaction Medium can contain hydrogel. In some embodiments, the hydrogel can be gelatin or sodium alginate. In some embodiments, the Reaction Medium can further contain yeast extract, peptone, D-glucose, and ampicillin. In some embodiments,

In some embodiments, in any of the methods described herein, the output signal can be based on spectrophotometric property or fluorometric property of the reporter entity. In some embodiments, the reporter entity can be a fluorescent protein. In some embodiments, the reporter entity can be a luminescent protein. In some embodiments, the luminescent protein can be luciferase. In some embodiments, the readout reagent can include luminescence substrate and luminescence buffer. In some embodiments, the output signal can be luminescence.

In some embodiments, any of the methods described herein can include at least one logical operator module. In some embodiments, a logical operator module of the at least one logical operator module can include the at least one sample object and two or more CUs of the at least one CU. In some embodiments, the at least one logical operator module can generate one or more output signals. In some embodiments, the at least one logical operator module can include a YES gate, an AND gate, a NAND gate, an OR gate, a NOR gate, a XOR gate, a XNOR gate, a NOT gate, or any combination thereof, wherein the two or more CUs comprise a first CU and a second CU. In some embodiments, the YES gate can include generating the one or more output signals only when both the first CU and the second CU can be interacting with the at least one sample object. In some embodiments, the AND gate can include generating the one or more output signals only when both the first CU and the second CU can be interacting with the at least one sample object. In some embodiments, the NAND gate can include suppressing or diminishing the one or more output signals when both the first CU and the second CU can be interacting with the at least one sample object. In some embodiments, the OR gate can include generating the one or more output signals when either the first CU or the second CU or when both the first CU and the second CU can be interacting with the at least one sample object. In some embodiments, the NOR gate can include generating the one or more output signals when both the first CU and the second CU cannot be interacting with the at least one sample object. In some embodiments, the XOR gate can include generating the one or more output signals when either the first CU or the second CU but not both CUs can be interacting with the at least one sample object. In some embodiments, the XNOR gate can include generating the one or more output signals when either both the first CU and the second CU or when both the first CU and the second CU can be interacting with the at least one sample object. In some embodiments, the NOT gate can include suppressing or diminishing the one or more output signals when the first CU can be interacting with the at least one sample object.

In some embodiments, in any of the methods described herein, the sample object independently can be a cell or a molecule.

In some embodiments, in any of the methods described herein, a CU of the at least one CU can be independently (i) associated with one or more surface-bound entities (SBEs); (ii) capable of recognizing a signal object (SO); (iii) capable of producing an SO; (iv) capable of degrading an SO; (v) capable of producing a change in a material property of the reaction medium; (vi) capable of producing another CU; or (vii) capable of changing its state following signal recognition or external influence.

In some embodiments, in any of the methods described herein, the CU can include a molecule or a cell. In some embodiments, the molecule can include a monomeric or multimeric molecule. In some embodiments, the molecule can include a polypeptide, a polypeptide derivative, a nucleic acid, or a nucleic acid derivative. In some embodiments, the polypeptide can be an antibody or an enzyme. In some embodiments, the cell can include a wildtype cell, a synthetic cell, or an engineered cell. In some embodiments, the cell can be a yeast cell. In some embodiments, the yeast cell can be an engineered yeast cell.

In some embodiments, in any of the methods described herein, the CU state change can detect cell specific apoptosis. In some embodiments, an SBE of the one or more SBEs can be a phosphatidylserine binding SBE. In some embodiments, the one or more SBEs can be cell-specific, antigen binding SBEs. In some embodiments, the CU of the at least one CU can be associated with the phosphatidylserine binding SBE, and one or more CUs of the at least one CU can be associated with the cell-specific, antigen binding SBEs. In some embodiments, the yeast cell can include Annexin strain, EGFR strain, and BAR1 strain. In some embodiments, the Annexin strain can include a polypeptide sequence having at least about 80% sequence identity to SEQ ID NO: 1. In some embodiments, the Annexin strain can include a polypeptide sequence of SEQ ID NO: 1. In some embodiments, the EGFR strain can include a polypeptide sequence having at least about 80% sequence identity to SEQ ID NO: 2. In some embodiments, the EGFR strain can include a polypeptide sequence of SEQ ID NO: 2. In some embodiments, the BAR1 strain can include a polypeptide sequence having at least about 80% sequence identity to SEQ ID NO: 3. In some embodiments, the BAR1 strain can include a polypeptide sequence of SEQ ID NO: 3.

In some embodiments, in any of the methods described herein, the CU can include at least one SBE of the one or more SBEs. In some embodiments, an SBE of the one or more SBEs can include a cell surface receptor, a transmembrane protein or a glycophosphatidylinositol (GPI)-anchored protein. In some embodiments, the SO can include a bio-generated entity. In some embodiments, the bio-generated entity can include a signaling molecule, a metabolite, a peptide, or a synthetic compound. In some embodiments, the signaling molecule can include a hormone, a cytokine, an interleukin, a chemokine, or a pheromone. In some embodiments, the SO can be produced by a CU of the at least one CU. In some embodiments, the SO can be not produced by a CU of the at least one CU. In some embodiments, the CU can form one or more computational clusters. In some embodiments, a force can be applied to the CU to form the one or more computational clusters. In some embodiments, the force can be a centrifugal force, a magnetic force, or an electrostatic force. In some embodiments, the centrifugal force can be a continuous centrifugal force. In some embodiments, a reaction tube including the at least one sample object, the Reaction Reagent, and the Reaction medium can be twisted around its z-axis to create the continuous centrifugal force.

In another aspect, provided herein is a system for biological computing, which can include: (a) at least one sample object derived from an input sample; (b) a Reaction Reagent including at least one computing unit (CU) configured to interact with the at least one sample object; (c) a Reaction Medium; and (d) a readout reagent including a reporter entity and a reporter buffer. In some embodiments, the system can further include one or more evaluation strips and a plate adopter. In some embodiments, the system can further include one or more 96-well plates and one or more microcentrifuge tubes. In some embodiments, the system can further include a thermocycler. In some embodiments, the system can further include a plate reader. In some embodiments, in any of the systems described herein, one or more computational clusters can be formed when the at least one CU interacts with the at least one sample object. In some embodiments, a force can be applied to form the one or more computational clusters. In some embodiments, the force can be a centrifugal force, a magnetic force, or an electrostatic force. In some embodiments, the centrifugal force can be a continuous centrifugal force. In some embodiments, in any of the systems described herein, a reaction tube including the at least one sample object, the Reaction Reagent, and the Reaction medium can be twisted around its z-axis to create the continuous centrifugal force.

In yet another aspect, provided herein is a kit for biological computing, which can include: (a) one or more reaction tubes including Reaction Reagent; (b) one or more master tubes including Reaction Medium; (c) a readout reagent including a reporter entity and a reporter buffer; and (d) an instruction for use of the kit. In some embodiments, the kit can further include one or more evaluation strips and a plate adapter. In some embodiments, the kit can further include a blank sample.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic illustration of the workflow of the presently described biocytometry platform. The presently described biocytometry is designed to identify a given cell immunophenotype using computing unit types, where each type corresponds to a pre-selected binding and signaling type. The platform is robust to handling and sample format, including peripheral blood, cell lines, and either cryopreserved or stabilized samples. The process begins by combining the computing unit with a sample. This is followed by an incubation period, and finally a readout that determines the target cell count.

FIG. 2 is a schematic illustration of an overview of the presently disclosed biocytometry systems and methods of using the systems. The system contains, inter alia, engineered cells that can bind to cells in an input sample and evaluate their immune profiles. If a cell matches the targeted profile, the engineered cells exit their dormant state and enter an activated state to produce easily measurable reporter signals.

FIG. 3 is a schematic illustration of the mechanism of the presently disclosed biocytometry system. Computing units display antibody mimetics on their surface, defined by their binding type. These computing units efficiently bind to cells in a suspension, guided by each cell's unique immunophenotype. Within each computing unit-cell complex, computing units secrete trace amounts of pheromone according to their signaling type. Target immunophenotypes yield positive feedback signaling patterns that reinforce signaling activity and ultimately switch computing units to an activated state. Computing unit activation is marked by the turning on of programmable reporter gene circuits, e.g., a luminescent reporter is secreted into the Reaction Medium for sensitive enumeration of target cells.

FIG. 4 is a graph illustrating detection of target cell subpopulation by the presently disclosed biocytometry systems and methods. The target cells (HaCaT, EpCAM⁺) and background cells (HL-60, CD69 dim) were combined at various concentrations, stained by primary antibodies, and measured by anti-Fc reagent of the present disclosure or anti-Fc HRP conjugate. The presently disclosed biocytometry system exhibited an all-or-nothing behavior rejecting low expressing background cells and recognizing all high expressing target cells equally, while the target signal strength was diminished as the background increased in the enzymatic method (Fc).

FIG. 5 is a graph illustrating quantification of spiked target cell subpopulation by the presently disclosed biocytometry and method. Target cell dilution series was measured by the presently disclosed biocytometry system (BC) and flow cytometry (FC). Six independent samples were prepared. Each sample included varying amounts of target cells (HaCaT, EpCAM⁺) spiked into peripheral blood mononuclear cell (PBMC) sample matrix. Excellent correlation with the dilution series was attained with both the presently disclosed biocytometry system (R=0.9961) and flow cytometry (R=0.9931) in low sensitivity measurements, with the presently disclosed biocytometry system having exceptional false positive rate characteristics (0 vs. 899 false positive events per million cells analyzed for the presently disclosed biocytometry system and flow cytometry, respectively).

FIG. 6 is a schematic illustrating a non-limiting exemplary workflow of the presently disclosed biocytometry systems, kits, and methods.

FIG. 7 is a graph illustrating enumeration of EGFR positive cells apoptotic cells in Ara-C induced samples.

FIG. 8A is an illustration of target cells labeled with ATTO425-Mal enable post-analysis count confirmation via microscopy.

FIG. 8B is an illustration of a 98-well plate. Combining well luminescence with microscopic counts yields the signal-to-noise ratio per target (SNRT), a measure of detection performance.

FIG. 8C is a graph of target cell spiking experiment. Samples tubes were spiked with 0-1000 HaCaT (EpCAM+) target cells and processed according to the standard protocol with anti-EpCAM YES gate computing units. Luminescence readout demonstrates strong correlation with the number of spiked targets (R=0.990, p≈0). The 95% confidence intervals are also shown (gray area).

FIG. 8D is a graph illustrating a threshold limit for positive classification of SNR=5, with signals from wells containing zero target cells consistently remaining below this limit. Positive wells with a single target cell demonstrate mean SNRT=27.1.

FIG. 8E is a graph illustrating that the presently described biocytometry exhibits exceptional sensitivity, accurately identifying samples containing as few as 1 target cell and maintaining consistent performance across a wide dynamic range (1 to 1,000 target cells/reaction).

FIG. 8F is a graph illustrating the evaluation of performance of the presently described biocytometry in detecting target cells using a dual-marker (EpCAM and EGFR). Sample tubes, containing 0-30 target cells derived from HL-60 (EpCAM−/EGFR−), HeLa (EpCAM−/EGFR+), or HaCaT (EpCAM+/EGFR+) cell lines, were analyzed using either anti-EpCAM, anti-EGFR YES gate computing units, or anti-EpCAM+EGFR AND gate computing unit per the standard protocol. The mean SNRT values obtained were 27.0 for anti-EpCAM YES gate, 23.0 for anti-EGFR YES gate, and 26.7 for anti-EpCAM+EGFR AND gate computing units, demonstrating comparable detection efficacy (ANOVA, P=0.298). Signals from samples with zero-count target cells, HL-60 (double negative), or HeLa (single positive) target cells consistently fell beneath the threshold for positive classification. This underscores the presently described biocytometry's discriminative ability in a multi-marker detection context, as confirmed by the results of Mann-Whitney U tests (zero-count: P=2.11×10-5, HL-60: P=1.26×10-7, HeLa: P=1.71×10-7). ****, P<0.0001; NS, non-significant.

FIG. 8G is a graph for assessing robustness of the presently described biocytometry across varying sample sizes. A fixed number (~30 cells/sample) of EpCAM+ HaCaT target cells were integrated into HL-60 background cells (EpCAM−, 0 to 1×10⁶/sample). Anti-EpCAM YES gate computing units were utilized for analysis. Mean SNRT values remained stable across escalating background cell counts, ranging from 31.3 to 24.2, as confirmed by statistical analyses (ANOVA, p=0.137; Mann-Whitney U, p<10⁻⁶). HL-60 cells alone yielded no detectable signal.

FIG. 8H is a graph for examining the influence of single-marker positive cells on dual-marker detection. Samples were configured with a consistent count of HaCaT target cells (EpCAM+/EGFR+, ~30 cells/sample) and varying numbers of HeLa cells (EpCAM−/EGFR+, 0 to 1×10⁴/sample), all against a uniform HL-60 cell background (EpCAM−/EGFR−, 1×10⁵ cells/sample). Utilizing anti-EpCAM/anti-EGFR AND gate computing units per standard protocols, the assay maintained stable performance across differing HeLa cell counts. Mean SNRT values ranged from 16.7 to 20.6 (ANOVA, p=0.280; Mann-Whitney U test, p<10⁻⁶). Importantly, the assay retained specificity, as samples containing solely HeLa cells did not produce signal.

FIG. 8I is ROC curves generated for each sample, both by background cell size—0, 1×10⁻⁴, 1×10⁵, 1×10⁶ cells—and by the number of single-positive cells—0, 1×10², 1×10³, 1×10⁻⁴. The corresponding AUC values were 0.939, 0.997, 1.000, 0.917 for the background cell sizes and 0.939, 0.997, 1.000, 0.917 for the single-positive cell counts, respectively.

FIG. 9A is graphs illustrating comparative analysis of the presently described biocytometry and flow cytometry. Samples with different HaCaT (EpCAM+) target cell quantities (0, 4, 16, 64, 256, 1024) were prepared in a range of sample matrices, including peripheral blood (PB), PBMCs, or primary cell culture (PCC), each containing approximately 5×10⁵ leukocytes. Samples (n=3 independent replicates) in a target cell dilution series were analyzed in parallel. The presently described biocytometry utilized anti-EpCAM YES gate computing units, adhering to the clinical workflow. For flow cytometry, samples were stained with AF647-conjugated anti-EpCAM antibody, following the standard rare cell quantification method. A strong correlation between the two methods and the target cell dilution series for samples containing high target cell counts (64-1024 spiked target cells, >10⁻⁴ sensitivity) were observed. For samples with lower target cell counts (0-64 spiked target cells, <10⁻⁴ sensitivity), flow cytometry analysis was impeded by its limit of blank (FC LOB), failing to quantify below this level. The Spearman's (R) is shown. Uncertainty in target cell spiking counts is highlighted (gray area).

FIG. 9B is a graph illustrating the presently described biocytometry (BC) consistently demonstrating a null limit of blank (LOB) across all sample types. In contrast, flow cytometry (FC) showed varying false positive rates among tested sample matrices—highest in peripheral blood (121 per million cells analyzed), followed by PBMCs (55 per million), and primary cell cultures (31 per million). Error bars represent 1 s.d.

FIG. 9C is a graph illustrating the evaluation of performance of the presently described biocytometry's performance in absolute quantification within complex sample matrices. Akin to FIG. 9A, microscopic examination was employed to validate the exact count of the target cells. The mean SNRT values for target cell detection—17.9 for peripheral blood, 21.6 for PBMCs, and 19.3 for primary cell cultures—illustrate consistent performance across all tested matrices (ANOVA, P=0.991). Moreover, the presently described biocytometry exhibited robust detection characteristics, as demonstrated by Mann-Whitney U test results (PB: P=3.72×10-6, PBMC: P=2.57×10-5, PCC: P=1.76×10-6). ****, P<0.0001; NS, non-significant.

FIG. 9D is ROC curves generated for each sample matrix, revealing AUC values of 1.000, 0.999, and 0.9997 for peripheral blood, PBMCs, and primary cell cultures, respectively. Error bars represent 1 s.d.

FIG. 10A is a graph illustrating that apoptotic rate in primary cell cultures increased post-heat shock treatment, with 12.3% confirmed as apoptotic.

FIG. 10B is microscopy images of Annexin V-FITC staining highlighting the morphological features of apoptotic cells.

FIG. 10C is a graph illustrating that the presently described biocytometry (BC) maintains a null LOB, in contrast to the escalated LOB observed with flow cytometry (FC).

FIG. 10D is a scatter plot analysis illustrating strong correlation for the presently described biocytometry (R=1.000, p≈0) and suboptimal performance for flow cytometry (R=0.829, p=0.042) in apoptotic primary cell cultures. The Spearman's (R) is shown. Uncertainty in target cell spiking counts is highlighted (gray area).

FIG. 10E is a graph illustrating that with fluorescently-labeled HaCaT cells used in apoptotic primary cell cultures (APCC), biocytometry registers a mean SNRT of 25.6.

FIG. 10F is graph illustrating strong correlation (R=0.872, p≈0) between estimated target cell counts in bone marrow and biocytometry readouts. The 95% confidence intervals are also shown (gray area).

FIG. 10G is a graph illustrating mean SNRT in bone marrow is 20.0, demonstrating comparable performance to other matrices.

FIG. 10H is ROC curve analysis yielding an AUC value of 1.000 in APCC and 0.996 in bone marrow. Error bars represent 1 s.d.

FIG. 11A is a correlation plot showing a strong relationship (R=0.975, p<10⁻⁵) between CD25+ events estimated by the presently described biocytometry system using anti-CD25 YES gate computing units and flow cytometry with TRITC-conjugated anti-CD25 antibodies.

FIG. 11B is a Bland-Altman plot indicating a mean bias of 0.25% and limits of agreement from −1.83% to 2.33% between the two methods (biocytometry and flow cytometry) for leukocyte activation assessment.

FIG. 11C is a scatter plot illustrating the enumeration of CD34+ hematopoietic stem cells (HSCs) in a dilution series derived from D0 and D5 samples during G-CSF-induced mobilization. Biocytometry achieves a perfect linear fit (R=1.000, p≈0), in contrast to the suboptimal performance of flow cytometry at low HSC concentrations. Coefficients of variation (CVs) are 18.4% for biocytometry and 24.9% for flow cytometry, with flow cytometry displaying a higher CV (45.2%) at the lowest HSC concentration.

FIG. 12A is graphs illustrating a noticeable decrease in the total count of EpCAM+ cells (utilizing the EpCAM kit), aligning with the anticipated apoptotic and subsequent necrotic changes in the culture, as corroborated by the EpCAM/Apoptosis kit readout.

FIG. 12B is a graph illustrating that a robust response to DTT induction for HCT cells was evident upon normalization of the signals from the EpCAM/Apoptosis kit with the numbers obtained with EpCAM kit.

FIG. 12C is a graph illustrating the apoptotic rate remains consistent irrespective of the JUR (EpCAM−) cell count. This confirmed that the EpCAM/Apoptosis kit's specificity for detecting apoptosis in HCT (EpCAM+) cells, as evidenced by the absence of measurable events in HCT-devoid samples.

FIG. 13A is a statistical model demonstrating that up to four types of computing units, each with unique antigen specificity, can be used to determine a target profile with over 99% sensitivity. The assumptions of the model are as follows: a single target can support up to 60 computing units, corresponding to a target with an approximate radius of 10 pm (typical of a small mammalian cell). Each computing unit is presumed to have an equal propensity for binding. It is also posited that at least six copies of a given computing unit type are required to reconstruct a profile, a figure that has been experimentally validated (data not shown).

FIG. 13B is an immunofluorescence image illustrating equal representation of computing unit types confirmed using the HaCaT cell line with the BIOS(EpCAM+EGFR) system. EpCAM-specific computing units were stained blue and EGFR-specific computing units were stained yellow. Binding conditions were optimized to ensure lower number of bound computing units, thus facilitating the differentiation of computing unit types. The scale bar indicates 10 pm.

FIG. 13C is a scanning electron microscopy image facilitating the count of computing units on a target cell surface.

FIG. 14A is a microscopy image of computing unit-cell complex formation for evaluating efficiency of SAG1 anchoring mechanisms. HaCaT cells (EpCAM+) were subjected to the standard biocytometry workflow using BIOS(EpCAM).

FIG. 14B is a microscopy image of computing unit-cell complex formation for evaluating efficiency of FLO11 anchoring mechanisms. HaCaT cells (EpCAM+) were subjected to the standard biocytometry workflow using BIOS(EpCAM). From a randomly selected subset of 10 cell-computing unit complexes per anchor type, comparative analysis revealed a substantial 652% enhancement in binding efficiency when employing the FLO11 anchor system.

FIG. 14C is a microscopy image illustrating that diverse set of binders can support the formation of computing unit-cell complexes. Microscopic visualization of complexes formed between BIOS utilizing anti-Her2 Affibody with HaCaT cells, anti-EGFR DARPin with HeLa cells, anti-CD45 scFv with HL-60 cells, and anti-CD71 antibody conjugate with Jurkat cells.

FIG. 15A is a graph of pheromone dose-response profiling of MATa-derived computing units investigated across wild-type background with a reporter module (BSX6239), wild-type with reporter and binding modules (BSX6235), engineered background incorporating these modules (sX875), and strain with the full complement of reporter, binding, and signaling modules (sX882). Enhanced pheromone sensitivity was evidenced by the substantial reduction in EC50 in the engineered strains.

FIG. 15B is plots of quantitative analysis of pheromone leakage and active production in computing units investigated across wild-type backgrounds with a reporter module (BSX6239, BSX6240), wild-type with reporter and binding modules (BSX6235, BSX6236), engineered backgrounds incorporating these modules (sX875, sX878), and strains with the full complement of reporter, binding, and signaling modules (sX882, sX884). The results indicated minimized pheromone leakage and maximized production upon activation in the engineered strains.

FIG. 16 is plots illustrating genetic modification and their effects on reporter fold-induction. The reporter fold-induction was measured for three different computing unit background strains to evaluate the effects of two genetic modifications: incorporation of pFIG1(I) and modification of the GPA1 subunit. For both modified strains, a significant increase in fold-induction was observed—a 10-fold increase for MATa-origin computing units and a 30-fold increase for MATa-origin computing units, with the pFIG1(I) implementation yielding the highest increase in reporter fold-induction for both mating types. The data presented are indicative of the ratio of reporter production measured at 180 minutes post-induction to the baseline reporter production without induction, across n=2 biological replicates for each strain.

FIG. 17A is a plot of assessment of assay media formulations on the detection efficacy of target HaCaT (EpCAM+) cells. Various media were tested, including YPD (the standard medium for yeast cultivation), Formulation 1 (comprising only suppressor biopolymers within a hydrogel matrix), Formulation 2 (containing only crowding biopolymers in a hydrogel matrix), and Formulation 3 (the hydrogel system employed throughout Examples of the present disclosure). Sample tubes containing 0-30 target cells derived from HaCaT (EpCAM+) cell lines were analyzed using BIOS(EpCAM) per the standard protocol. The mean SNRT values obtained were 3.0 for YPD, 5.1 for Formulation 1, 12.8 for Formulation 2, and a notably higher 20.9 for Formulation 3.

FIG. 17B is a graph illustrating that the secreted protease in BAR1 computing units effectively degraded alpha-factor, raising the activation threshold of the biosensors.

FIG. 17C is a plot of comparative analysis of assay performance using EpCAM-specific computing units: the wild-type S288C strain (wt001), a strain harboring a partial suite of chromosomal modifications (wt002), and the fully genetically modified BIOS(EpCAM) strain. The BIOS(EpCAM) strain outperformed with a mean SNRT value of 19.2, in contrast to 0.1 for wt001 and −0.2 for wt002. Error bars represent 1 s.d. ****, P<0.0001; NS, nonsignificant. A line at SNR=5 delineates the upper limit for negative samples.

FIG. 18A is an immunofluorescence image of EpCAM and EGFR expression in HaCaT cells visualized using anti-EpCAM-FITC and anti-EGFR-AF555 antibodies, respectively. Microscopic examination confirmed a robust signal with correct membrane localization on HaCaT cells.

FIG. 18B is a plot illustrating the efficacy of different BIOS computing units compared in assays with corresponding target cells: BIOS(CD34) with KG1a cells, BIOS(CD138) with RPMI8226 cells, and BIOS(EGFR) with HeLa cells. These combinations yielded consistent assay performance, with mean SNRT values of 26.7 for KG1a, 19.5 for RPMI8226, and 24.3 for HeLa cells. Error bars represent 1 s.d. ****, P<0.0001; NS, nonsignificant. A line at SNR=5 delineates the upper limit for negative samples.

FIG. 19 is Youden plots from a multicenter study display high interfacility concordance in quantifying cell populations larger than 0.5%. For cell counts approaching the detection limit of flow cytometry (−0.1% CD34+ cells), there was a notable increase in the coefficient of variation, indicating variability and challenges in reproducibility.

### DETAILED DESCRIPTION OF THE DISCLOSURE

The presently described biocytometry systems, kits, and methods can contain, inter alia, engineered or synthetic cells that can perform tasks normally executed by complex instruments. The engineered or synthetic cells of the present disclosure can bind to cells in an input sample and evaluate their profiles, allowing analysis of as many cells in a day as the currently available technologies currently do in a year, with unprecedented levels of sensitivity and specificity. Furthermore, the presently described biocytometry systems, kits, and methods are highly modular. By mixing and matching the types of engineered or synthetic cells, the target profile can be modified or customized as desired by users by using logic gates, details of which are described further below. The adaptability of the present disclosure allows for analysis of anything from e.g., rare epithelial cells in samples of lysed blood to e.g., apoptotic T-cells in primary cell cultures. Various aspects and embodiments of the present disclosure are described in greater details below.

The following descriptions and examples illustrate embodiments of the present disclosure in detail. Although the present disclosure has been described in some details by way of illustration and example for purposes of clarity and understanding, it will be apparent that certain changes and modifications can be practiced within the scope of the appended claims.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment. It is to be understood that the present disclosure is not limited to the particular embodiments described herein and as such can vary. Those of skill in the art will recognize that there are variations and modifications of the present disclosure, which are encompassed within its scope.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise.

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated cases, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a”, “an”, and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C”. The term “or” can be used conjunctively or disjunctively, unless the context specifically refers to a disjunctive use.

Furthermore, the use of the term “including” as well as other forms, such as “include”, “includes” and “included”, is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Where particular values are described in the application and claims, unless otherwise stated the term “about” meaning within an acceptable error range for the particular value should be assumed.

“Sequence identity” refers to the extent to which two optimally aligned polynucleotides or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides or polypeptides in a linear polynucleotide or polypeptide sequence of a reference (“query”) polynucleotide molecule (or its complementary strand) or polypeptide molecule as compared to a test (“subject”) polynucleotide molecule (or its complementary strand) or polypeptide molecule when the two sequences are optimally aligned. In some embodiments, “percent identity” can refer to the percentage of identical amino acids in an amino acid sequence.

The term “fragment” or “variant” refers to any functional fragment, variant, derivative or analog of a polynucleotide, polypeptide or biomolecule that possesses an in vivo or in vitro activity that is characteristic of the polynucleotide, polypeptide or biomolecule. In some embodiments, the fragment, variant or analog has a length equal to 10%, 20%, 30%, 40%, 50%, 60%, 70%, 80% or 90% or greater of the length of the polynucleotide, polypeptide or biomolecule. Functional expression of the fragment or variant can be easily assayed by the person of ordinary skill in the art by testing activity and the ability to manufacture products as described herein.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Methods of the Disclosure

Single-cell research sits at the intersection of the current understanding of health and disease, offering unprecedented insights into cellular mechanisms and creating opportunities throughout the pharmaceutical value chain. However, the swift translation of these discoveries into real-world applications is hampered by significant limitations of current technologies. Chief among these obstacles are the sensitivity and scalability of cell identification. The former is integral for enabling accurate single-cell analysis of more than the most ubiquitous cell types, while the latter becomes paramount when considering economics and the volume of samples necessary for validation of cellular biomarkers established via single-cell profiling. Standard methods in use today include variations and iterative improvements in traditional imaging and flow cytometry solutions. However, the sensitivity and scalability of these methods are fundamentally limited by data noise and operational requirements. Presented herein are highly adaptable and modular novel methods (biocytometry) of overcoming these limitations.

The presently described biocytometry system enables rapid analysis of an input sample with unprecedented levels of sensitivity and specificity, allowing analysis of as many cells in a day as the currently available technologies currently do in a year. For example, by mixing and matching different types of engineered cells comprising one or more computing units (CUs), the target profile can be modified and customized in any way the user desires. Modular logical operator modules can also be customized based on the target profile. The logical operator modules can receive one or more input signals (e.g., sample objects) and can generate one or more output signals (e.g., output signal objects (SOs)) based on the specified modules.

For example, the sample objects (derived from the input sample) can be mixed with the Reaction Reagent and Reaction Medium to allow the sample objects to interact with the CUs of the present disclosure, thereby forming one or more computational clusters and generating output signals. If the sample objects (e.g., cells) match the targeted profile, the engineered cells exit their dormant state and enter an activated state, wherein they can produce easily measurable output signals (e.g., reporter signals). The presence or absence of output signals can be read and/or quantified by use of the readout reagent. The target profile can be modified in any way the user desires by using the logical operator module of the present disclosure. In a non-limiting example, a two-input “AND” gate requires two specifics e.g., antigens to both be present on the bound cell to trigger a positive readout. Various aspects and embodiments of the logical operator modules are described in greater detail below.

Accordingly, in one aspect, provided herein is, inter alia, a method of detecting presence or absence of an output signal indicative of a characteristic of an input sample, the method comprising (a) contacting at least one sample object derived from the input sample with a Reaction Reagent comprising at least one computing unit (CU) and with a Reaction Medium, wherein the at least one CU can be configured to interact with the at least one sample object; (b) incubating (a) for a sufficient amount of time in an incubator for the output signal to be generated; (c) adding a readout reagent comprising a reporter entity to (b), wherein the reporter entity generates the output signal; and (d) measuring the output signal, wherein the output signal can be directly proportional to the amount of the at least one sample object derived from the input sample, thereby detecting the presence or absence of the output signal indicative of the characteristic of the input sample. In some embodiments, the contacting in step (a) comprises first (i) adding the at least one sample objects to the Reaction Reagent and then (ii) adding the Reaction Medium to (i). Non-limiting exemplary characteristics of the input sample can be apoptosis, activation, suppression, anergy, or differentiation. In some embodiments, the characteristic can be cell-specific apoptosis.

In another aspect, provided herein is a method of quantifying the level of a target object in an input sample, the method comprising (a) contacting at least one sample object derived from the input sample with a Reaction Reagent comprising at least one computing unit (CU) and with a Reaction Medium, wherein the at least one CU can be configured to interact with the at least one sample object; (b) incubating (a) for a sufficient amount of time in an incubator for the output signal to be generated; (c) adding a readout reagent comprising a reporter entity to (b), wherein the reporter entity generates the output signal; and (d) measuring the output signal, wherein the output signal can be directly proportional to the amount of the at least one sample object derived from the input sample, thereby quantifying the output signal indicative of the level of the target object in the input sample. In some embodiments, the contacting in step (a) comprises first (i) adding the at least one sample objects to the Reaction Reagent and then (ii) adding the Reaction Medium to (i).

In yet another aspect, provided herein is a method of diagnosing a disease or condition individual, the method comprising (a) contacting at least one sample object derived from the input sample (derived from the individual) with a Reaction Reagent comprising at least one computing unit (CU) and with a Reaction Medium, wherein the at least one CU can be configured to interact with the at least one sample object; (b) incubating (a) for a sufficient amount of time in an incubator for the output signal to be generated; (c) adding a readout reagent comprising a reporter entity to (b), wherein the reporter entity generates the output signal; and (d) measuring the output signal, wherein the output signal can be directly proportional to the amount of the at least one sample object derived from the input sample, thereby detecting the presence or absence of the output signal indicative of the presence or absence of the disease in the input sample. In some embodiments, the contacting in step (a) comprises first (i) adding the at least one sample objects to the Reaction Reagent and then (ii) adding the Reaction Medium to (i).

In any of the methods described herein, the sufficient amount of time in step (b) can be about 2 hours, about 2.1 hours, about 2.2 hours, about 2.3 hours, about 2.4 hours, about 2.5 hours, about 2.6 hours, about 2.7 hours, about 2.8 hours, about 2.9 hours, about 3 hours, about 3.1 hours, about 3.2 hours, about 3.3 hours, about 3.4 hours, about 3.5 hours, about 3.6 hours, about 3.7 hours, about 3.8 hours, about 3.9 hours, about 4 hours, about 4.1 hours, about 4.2 hours, about 4.3 hours, about 4.4 hours, about 4.5 hours, about 4.6 hours, about 4.7 hours, about 4.8 hours, about 4.9 hours, about 5 hours, about 5.1 hours, about 5.2 hours, about 5.3 hours, about 5.4 hours, about 5.5 hours, about 5.6 hours, about 5.7 hours, about 5.8 hours, about 5.9 hours, or about 6 hours. In some embodiments, the sufficient amount of time in step (b) can be about 3 hours. In some embodiments, the sufficient amount of time in step (b) can be about 3.5 hours. In some embodiments, the sufficient amount of time in step (b) can be about 4 hours. In some embodiments, the sufficient amount of time in step (b) can be about 4.5 hours. In some embodiments, the sufficient amount of time in step (b) can be about 5 hours.

Various aspects and embodiments of the presently disclosed biocytometry methods are explained in greater details below. Below section headings are for organizational purposes only and are not to be construed as limiting the subject matter described.

### Biocytometry

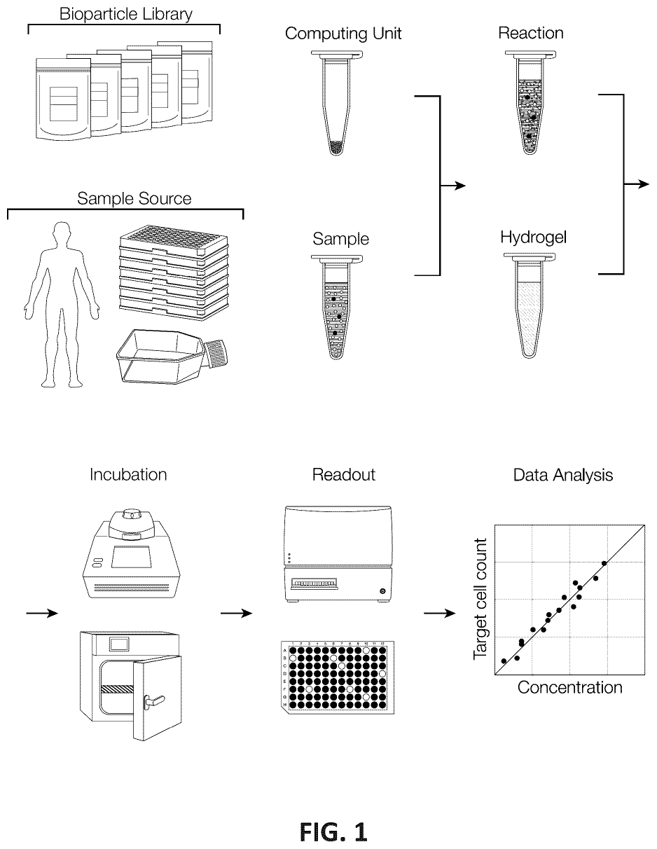

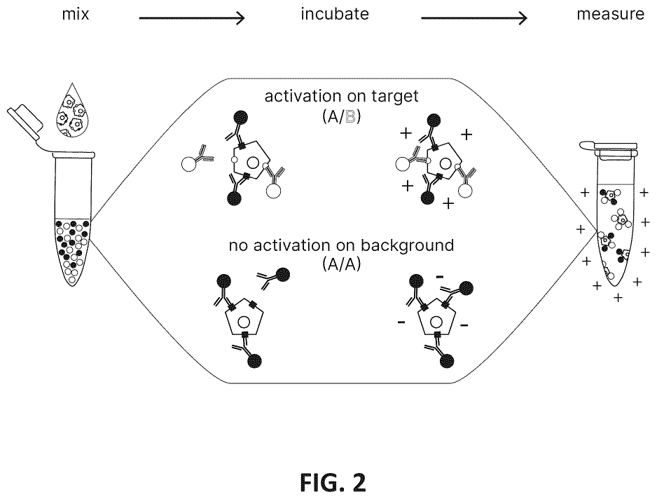

Presently described herein is biocytometry, a novel platform for multiparametric immunophenotyping in suspension. Biocytometry is a novel solution for target cell identification based on engineered computing units. Echoing natural processes of cellular identification, biocytometry enables the massively parallel identification of target cell immunophenotypes in suspension. By avoiding the limitations of traditional instruments and molecular probes, it adopts a streamlined workflow that can be integrated seamlessly with established analytical pipelines (FIGS. 1-3). The standard workflow can implement basic mixing and incubation steps combined with high-throughput spectrophotometry to easily accommodate the simultaneous measurement of a wide range of immunophenotypes. The presently described biocytometry system has been validated on a diverse set of immortalized cell lines, such as HeLa, HaCaT, KG1a, and Jurkat, which exhibit a diverse set of surface markers, including EGFR, EpCAM, CD34, CD25, and phosphatidylserine (see Examples infra).

A hallmark of biocytometry can be multiparametric cell identification, whereby a target cell type can be identified by a combination of surface markers satisfying a logic statement (i.e., logical operator modules, such as an AND, OR, or YES gate). In vitro data integration can be the cornerstone of biocytometry, enabling massively parallel analysis and consequently significant improvements in both sensitivity and scalability. Sensitivity of the presently described biocytometry is validated extensively in Examples infra, which also describe extensive comparative analysis between biocytometry and flow cytometry. In some embodiments, biocytometry can be equivalent to flow cytometry in lower-sensitivity quantifications (e.g., >10⁻⁴ sensitivity) and outperformed flow cytometry in higher-sensitivity quantifications (e.g., <10⁻⁴ sensitivity), achieving the theoretical limit of detection. In some embodiments, the sensitivity and specificity of biocytometry can remain undiminished when applied to challenging sample matrices (e.g., apoptotic primary cell cultures and bone marrow) (see Example 10). The presently described biocytometry system can be particularly effective in applications that demand high-sensitivity detection, such as, but not limited to, an enumeration of hematopoietic stem cells, CAR-T cells, or distinct immune cell clonotypes. The superior sensitivity and scalability of biocytometry methods are demonstrated across a diverse set of sample types in Examples infra, ranging from 2D cell cultures to bone marrow aspirates, through the successful identification of 12 distinct cell types.

Biocytometry, as described herein, introduces a divergent analytical framework, a potential milestone, that can enhance high-sensitivity cell analysis amidst ongoing reproducibility issues. The marked improvements over traditional cell analysis methods, such as flow cytometry, include: (i) biocytometry can perform massively parallel integration of cell-surface marker profiles in vitro, allowing the simultaneous measurement of thousands of assays; (ii) biocytometry can leverage cell-cell interactions over molecule-cell interactions, mitigating nonspecific binding and approaching the theoretical sensitivity limits; and (iii) biocytometry can utilize a streamlined homogeneous assay workflow, ensuring highly sensitive and reproducible sample evaluation, which can eliminate the necessity for specialized training and broaden the accessibility of cell analysis.

As described in detail in Examples infra, technical validation of biocytometry can reveal strong alignment with established technologies over a wide dynamic range while supporting multiparametric identification down to a single cell within large sample sizes. In some embodiments, HaCaT cells, an immortalized keratinocyte model characterized by the expression of EpCAM and EGFR surface markers that is critical to both diagnostic and prognostic applications, can be utilized in experiments validating biocytometry. In some embodiments, cell enumeration assay can yield cell counts in concordance with microscopy observation (Examples 2 and 8). In some embodiments, biocytometry can accurately identify samples containing as little as a single target cell and maintain consistent performance across a wide dynamic range (for example, 1 to 1,000 target cells/sample). In some embodiments, multiparametric identification via e.g., an AND gate for EpCAM and EGFR surface markers can successfully detect e.g., HaCaT cells (EpCAM+EGFR+), while control lines e.g., HeLa (EpCAM-EGFR+) and e.g., HL-60 (EpCAM-EGFR−) can generate signals indistinguishable from those of negative samples. In some embodiments, a sensitivity threshold for routine assessment can be established at e.g., 1×10⁻⁶, which is well beyond the conventional limit of 1×10⁻⁴ set by flow cytometry. As demonstrated in Example 3, the efficacy of multiparametric identification in biocytometry can be uncompromised by the presence of cells with partial target immunophenotypes, even when they constitute e.g., up to 10% of the sample.

As demonstrated in detail in Examples infra, biocytometry is compatible with clinically relevant sample types, achieving superior sensitivity and specificity over conventional flow cytometry. For example, a comparative study can utilize e.g., HaCaT cells spiked into various sample matrices, such as, but not limited to, peripheral blood, cryopreserved PBMCs, and primary cell cultures (Example 6). Furthermore, biocytometry can reliably maintain a null limit of blank (LOB) in all sample matrices, providing a robust framework for monitoring of cellular biomarkers, which can be crucial for understanding disease dynamics and tailoring more precise treatments. Example 6 demonstrates that flow cytometry analysis is constrained by its LOB for samples containing low target cell concentrations (e.g., 0-64 cells/million total cells; <10⁻⁴ sensitivity), while biocytometry can reliably maintain a null LOB in all sample matrices, providing a robust framework for monitoring of cellular biomarkers, which is crucial for understanding disease dynamics and tailoring more precise treatments.

Biocytometry is far more versatile than flow cytometry, especially when working with notoriously challenging sample matrices, such as, but not limited to, apoptotic primary cell cultures and bone marrow aspirates, complicated by high levels of stromal components and debris and by limited sample availability (Example 10). For example, flow cytometry shows a significant LOB increase by 464% for apoptotic samples, while biocytometry exhibits no such vulnerability and maintains nominal target cell quantification metrics. Similarly, biocytometry can achieve a near-perfect area under the ROC curve (AUC) in ROC analysis with bone marrow samples. In some embodiments, biocytometry can outperform flow cytometry and exhibit a strong linear relationship with sample dilution series (e.g., for enumeration of hematopoietic stem cells), while flow cytometry exhibits a weaker correlation (Example 8).

Conventional methods assessing apoptosis, such as Caspase-based assays and MTT tests, yield limited sensitivity and quantify only aggregate apoptosis levels. Unlike these conventional methods, biocytometry can assess cell-specific apoptosis in mixed cell cultures, making it possible to study apoptosis in complex biological systems (Example 11). For example, through multiparametric identification, apoptotic rate of e.g., HaCaT cells mixed with e.g., Jurkat cells can be discerned. This marks the first reported use of a homogeneous assay for targeted apoptosis quantification. Through significant improvements across multiple performance metrics, biocytometry is in a unique position to reshape key sectors in diagnosis and therapy. Specifically, biocytometry is well suited for facilitating the monitoring of rare premalignant subpopulations in conditions, such as, but not limited to, smoldering multiple myeloma (SMM), enhancing the detection of circulating tumor cells (CTCs), advancing pharmacokinetic assessments in CAR-T therapeutic regimens, and improving the accuracy of minimal residual disease (MRD) evaluations. Furthermore, biocytometry can facilitate the detection of rare clonotypes expressing unique TCR and BCR repertoires, making vaccine efficacy studies more reliable. Biocytometry can also significantly increase the throughput of cell-specific readouts for in vitro drug discovery using more human-like samples, a high priority area enabling a shift from animal models. Furthermore, biocytometry is not limited in target size and extends naturally to the quantification of cell-cell interactions, spheroids, and other irregular objects. Biocytometry can also enable the measurement of other data types that are inaccessible by traditional methods.

### Input Samples

Input samples can be collected from various origins, e.g., biological origin. In some embodiments, the input sample can be biological. In some embodiments, the biological input sample can comprise a biological fluid (e.g., whole blood, serum, plasma, sputum, urine, saliva, nipple aspirate, ductal lavage, vaginal fluid, nasal fluid, ear fluid, gastric fluid, cerebrospinal fluid, sweat, pericrevicular fluid, semen, prostatic fluid, feces, cell lysate, or tears). In some embodiments, the biological input sample can comprise a tissue samples (e.g., hair, skin, or biopsy material). In some embodiments, the biological input sample can comprise an enriched biological material (e.g., various cell types or exosomes). In some embodiments, the input sample can be a biological sample derived from a subject or an individual.

In some embodiments, the presently described methods can be compatible with whole blood, stabilized leukocyte fractions, isolated PBMCs, cryogenically stored PBMCs, and primary cell cultures. In some embodiments, the recommended cell resuspension solution can be 1×DPBS containing 0.1% gelatin w/v. In some embodiments, standard cultivation media can also be compatible.

In some embodiments, an input sample should not exceed given limits in a standard reaction tube. In some embodiments, sample volume can be about 10 μL, about 20 μL, about 30 μL, about 40 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 210 μL, about 220 μL, about 230 μL, about 240 μL, about 250 μL, about 260 μL, about 270 μL, about 280 μL, about 290 μL, about 300 μL, about 310 μL, about 320 μL, about 330 μL, about 340 μL, about 350 μL, about 360 μL, about 370 μL, about 380 μL, about 390 μL, about 400 μL, about 410 μL, about 420 μL, about 430 μL, about 440 μL, about 450 μL, about 460 μL, about 470 μL, about 480 μL, about 490 μL, or about 500 μL In some embodiments, sample volume can be 100 μL.

In some embodiments, the total number of cells (passive background) of the input sample can be about 100,000 cells, about 200,000 cells, about 300,000 cells, about 400,000 cells, about 500,000 cells, can be about 600,000 cells, about 700,000 cells, about 800,000 cells, about 900,000 cells, or about 1,000,000 cells. In some embodiments, the total number of cells (passive background) of the input sample can be about 1 million cells, about 1.5 million cells, about 2 million cells, about 2.5 million cells, or about 3 million cells. In some embodiments, the total number of cells (passive background) of the input sample can be about 1 million cells.

In some embodiments, cells expressing at least one target antigen (active background) can be about 1%, about 2%, about 3%, about 4%, about 5%, about 10%, about 15%, about 20%, about 25%, about 30%, about 35%, about 40%, about 45%, about 50% of the passive background. In some embodiments, cells expressing at least one target antigen (active background) can be about 10% of the passive background.

In some embodiments, cells expressing all target antigen (target cells) can be about 1%, about 2%, about 3%, about 4%, about 5%, about 10%, about 15%, about 20%, about 25%, about 30%, about 35%, about 40%, about 45%, about 50% of the active background. In some embodiments, cells expressing all target antigen (target cells) can be 10% of the active background.

### Sample Objects

The sample objects are entities derived from the input sample, such that the CUs of the present disclosure, configured to interact with the sample objects, can generate an output signal indicative of a characteristic of the input sample.

In some embodiments, a sample object can comprise a cell. In some embodiments, the sample object comprises a cell associated with a surface-bound entity (SBE), which is further explained below. In some embodiments, the sample object can associate with an SBE of a CU. In some embodiments, a sample object can produce a signal object (SO), which is further explained below. In some embodiments, a sample object can degrade a signal object (SO). In some embodiments, a sample object can recognize an SO. In some embodiment, such recognition can lead to the sample object modulating its ability of producing or degrading an SO. In some embodiments, sample objects can be individual entities or clusters formed through aggregation of entities. For example, sample objects can be cells, and cell clusters can be formed due to specific cell-cell interactions, wherein the formation of cell clusters can signify cytotoxicity, adherence, and/or differentiation of cells or cell lineage.

In some embodiments, a sample object can comprise a molecule. In some embodiments, the molecule can comprise a wide array of molecules produced by the input sample. In some embodiments, the molecule can be a metabolite and/or its related compounds. In some embodiments, the molecule can be a short peptide and/or its derivatives. In some embodiments, the molecule can be a pheromone and/or its derivative compounds. In some embodiments, the molecule can be a signaling molecule, which can include a wide array of mammalian hormones, cytokines, interleukins, and/or chemokines.

In some embodiments, sample objects from the input sample or within the set of computing entities can be pre-treated prior to any of the presently described methods. In some embodiments, pre-treatment can slow or promote metabolic processes through external influence (e.g., temperature change) or chemical treatment (e.g., metabolite or inducer supplement). In some embodiments, pre-treatment can also expose or conceal object surfaces. In some embodiments, SBEs can be occluded by nonspecific layers (e.g., polysaccharide, glycoprotein layers). Such layers can be removed by appropriate enzymatic or chemical pre-treatment. In some embodiments, sample objects can comprise background entities that can hinder recognition of target objects. Erroneous clustering with background objects can be minimized by pre-treatment of the objects with elements that can interact non-specifically with surface entities and thereby fill the residual binding capacity. In some embodiments, blocking entities can inhibit non-specific binding (passive and covalent) between SBEs or between surfaces. Such blocking entities do not exhibit cross-reactivity with SBEs, and thus should not disrupt the sample objects.

### Reaction Reagent

The Reaction Reagent is a composition comprising at least one computing unit (CU), wherein each CU of the at least one CU can be configured to interact with a sample object derived from an input sample such that an output signal indicative of a characteristic of the input sample can be generated. In some embodiments, the Reaction Reagent contains hydrogel. In some embodiments, the hydrogel can be gelatin. In some embodiments, the Reaction Reagent can further contain one or more additional components, such as yeast extract, peptone, D-glucose, Dulbecco's phosphate-buffered saline, D-(+)-Trehalose dihydrate, and/or skim milk.

The components of the Reaction Reagent can be configured such that one or more computational clusters can be formed upon coming in contact with the at least one sample object, wherein each computational cluster comprises, independently, the at least one CUs. For example, without limitation, by mixing and matching the types of CUs (e.g., engineered or synthetic cells), the target profile can be modified as desired by users.

In some embodiments, any components of the Reaction Reagent described herein can be formulated with acceptable excipients, such as carriers, solvents, stabilizers, diluents, etc., depending upon a customized combination of CUs and target profile. Suitable excipients can include, for example, carrier molecules that include large, slowly metabolized macromolecules such as proteins, polysaccharides, polylactic acids, polyglycolic acids, polymeric amino acids, amino acid copolymers, and inactive viral particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

In some embodiments, the Reaction Reagent can be dehydrated and rehydrated prior to or upon contacting the at least one sample object from the input sample.

In some embodiments, the Reaction Reagent can be stored up to about 1 weeks, up to about 2 weeks, up to about 3 weeks, up to about 4 weeks, up to about 5 weeks, up to about 6 weeks, up to about 7 weeks, up to about 8 weeks, up to about 9 weeks, or up to about 10 weeks at room temperature without loss of signal. In some embodiments, the Reaction Reagent can be stored up to about 2 weeks at room temperature without loss of signal. In some embodiments, the Reaction Reagent can be stored up to about 1 weeks, up to about 2 weeks, up to about 3 weeks, up to about 4 weeks, up to about 5 weeks, up to about 6 weeks, up to about 7 weeks, up to about 8 weeks, up to about 9 weeks, up to about 10 weeks, up to about 11 weeks, up to about 12 weeks, up to about 13 weeks, up to about 14 weeks, up to about 15 weeks, or up to about 16 weeks at 4° C. without loss of signal. In some embodiments, the Reaction Reagent can be stored up to about 8 weeks at 4° C. without loss of signal.

### Computing Units (CUs)

A computing unit is an object that has the potential to affect the informational output (e.g., output signal) of the presently described methods. Informational output can be affected in a number of different ways. For example, a CU can be an object with a classified surface-bound entity (SBE) profile, thereby mediating object association. In another example, a CU can be an object that can produce other CUs or signal objects (SOs), thereby affecting the informational output of a customized method. In yet another example, a CU can be an object that can recognize SOs, thereby affecting the memory capacity of the presently described system.

In some embodiments, according to any of the methods described herein, a CU of the at least one CU can be, independently, (i) associated with one or more surface-bound entities (SBEs); (ii) capable of recognizing a signal object (SO); (iii) capable of producing an SO; (iv) be capable of degrading an SO; (v) capable of producing a change in a material property of the reaction medium; (vi) capable of producing another CU; or (vii) capable of changing its state following signal recognition or external influence.

In some embodiments, a CU can be capable of producing a change in a material property of the Reaction Medium, which is further described below, of the present disclosure upon coming in contact with the Reaction Reagent comprising CUs. In some embodiments, a CU can be capable of producing a reporter entity, such as, but are not limited to, fluorescent proteins (e.g., GFP, RFP, YFP, or CFP), luminescent proteins (e.g., luciferase, such as, but are not limited to, Gaussia princeps luciferase (GLuc), Metridia longa luciferase (MLuc), Renilla renformis luciferase (RLuc), Cypridina noctiluca luciferase (CLuc)), enzymes (e.g., beta-lactamase, beta-galactosidase, SEAP), any functional fragments or variants thereof. In some embodiments, the material property of the Reaction Medium can be an optical property, an electrical property, or a thermal property.

A CU can comprise non-native elements, native elements in non-native locations, or other alterations to native elements. Introduced elements can include, but are not limited to, signal object generators, SBEs, reporter molecules, regulatory sequences, genetic selection markers, other types of non-genetic markers (e.g., magnetic, immunological), reference reporter molecules, enzyme coding genes (e.g., protease, kinase, phosphatase), etc. In some embodiments, modifications can be performed by introducing genetic changes at select chromosomal regions. Chromosomal modifications can either introduce new genetic elements at one or more desired loci or modify native elements (promoters, degradation tags, termini tags, transposon sites, etc.) at native loci. In some embodiments, modifications can be performed by introducing additional genetic material, e.g., plasmids or synthetic chromosomes.

In some embodiments, a CU can be a wildtype cell (e.g., of bacterial, yeast, mammalian origin) or an engineered or synthetic cell (e.g., any genetically engineered cell). In some embodiments, a CU can be an engineered or synthetic cell based on or derived from a yeast cell. In some embodiments, suitable yeasts can include, but are not limited to, Pichia pastoris, Saccharomyces cerevisiae, Arxula adeninivorans (Blastoborys adeninivorans), Candida boidinii, Hansenual polymorpha (Pichia angusta), Kluveromyces lactis, Yarrowia lipolytica etc. In other embodiments, a CU can be monomeric or multimeric molecule (e.g., a polypeptide, polypeptide derivative, nucleic acid).

In some embodiments, a CU can be a cell comprising an SBE covalently attached to an anchor present within the cell. In some embodiments, the SBE can be covalently attached to the anchor by a linker. In some embodiments, the linker can comprise a repeat motif. In some embodiments, the linker can facilitate accessibility of the SBE and/or increases an effective contact area between a sample object and the CU upon coming in contact with the Reaction Medium.

In some embodiments, a CU can be a cell displaying SBEs on its outermost surface (e.g., a cell wall or membrane). In such case, the SBEs and the cell can be produced separately and subsequently associated by standard practices, e.g., through immunological labeling. Alternatively, SBEs can be produced by the CU itself and tethered or anchored on the surface. A SBE can be tethered to the surface by interaction with surface anchored proteins. Alternatively, a SBE can be anchored to the surface as a fusion polypeptide with a surface anchor moiety that interacts either with the membrane (e.g., by hydrophobic interaction with membrane lipids) or the cell wall (e.g., covalent bonding to cell wall polysaccharides or polypeptides).

In some embodiments, a CU can be a molecule (e.g., a monomeric or multimeric molecule). In some embodiments, the molecule can include, without limitation, a polypeptide, a polypeptide derivative, a nucleic acid, and/or a solid support. In some embodiments, a CU can comprise a polypeptide. In some embodiments, the polypeptide can be an antibody, e.g., a bispecific antibody (BsAb). In some embodiments, the polypeptide can be an enzyme. In some embodiments, the method can further comprise an agent, which the enzyme can convert into a signal object (SO). In some embodiments, a CU can comprise a solid support. In some embodiments, the solid support can be a functionalized bead.

In some embodiments, a CU can be a molecule with a single SBE or multiple SBE moieties of the same or different specificities. This can include a collection of immunoglobulins, their derivatives (e.g., scFv, Fab, diabody, etc.), or similar binding entities with some level of molecular specificity (e.g., DARPins, TALENS, antigens, nucleic acids).

In some embodiments, a first CU of the at least one CU can be capable of producing a second CU, wherein the second CU can be of the same type as the first CU or of a different type than the first CU.

In some embodiments, a first CU of the at least one CU can be associated with a first SBE and a second CU of the at least one CU can be associated with a second SBE. In some embodiments, the first SBE and second SBE can be capable of forming a complex comprising the first SBE and second SBE.

CUs can be classified by the types of SBEs they expose to the Reaction Medium. Each SBE can be typed by how it can be recognized (e.g., by van der Waals forces, hydrogen bonds, hydrophobic and/or ionic interactions). Presentation of SBEs can be either constant, spontaneous, or induced (e.g., initiated following detection of signals transmitted in the medium or following direct interaction with objects). CUs can be classified at each point in time. A CU's class can change in time as the CU's SBE profile changes. A CU can be called a target object at a given point in time if its current SBE profile belongs to one of the predetermined classes. If two target objects have statistically similar SBE profiles, they can be assigned to the same target object class.

### Surface-Bound Entities (SBEs)

SBEs can mediate object association either directly or through intermediate objects. In some embodiments, a SBE profile can define the CU class that subsequently serves in constructing the methods and systems that perform particular computing functions. In some embodiments, the SBE profile can be constant in time, change spontaneously (e.g., by random change of internal state), change as a result of an internal state change (e.g., caused by an internal engineered mechanism), or change as a result of induction (e.g., by chemical, temperature, light, or electromagnetic changes). In some embodiments, an SBE can have a dual purpose of (1) binding to a CU to a sample object; and (2) recognizing an SO.

In some embodiments, a SBE can be an antigen on the cell surface (e.g., cell surface receptor). In some embodiments, the antigen can be a disease-associated antigen (e.g., cancer-associated antigen). In some embodiments, the SBE can be a marker indicating a state of the sample object (e.g., differentiation factors or clusters of differentiation (CDs)). In some embodiments, the SBE can be a biological signal (e.g., MHC, MHC epitope complex, or glycocalyx). In some embodiments, the SBE can be a marker indicating an activity of the sample object (e.g., receptors, receptor ligand complexes, or ion channels, and their modified forms). In some embodiments, the SBE can be a pathogenic marker (e.g., glycoproteins or lectins). In some embodiments, the SBE can be a synthetically produced molecule (e.g., surface tags, displayed epitopes, or conjugated molecules).

In some embodiments, a SBE can be covalently attached to a surface of a CU. In some embodiments, the SBE can be covalently attached to the surface of the CU by a linker. In some embodiments, the linker can comprise a repeat motif. In some embodiments, the linker can be a polypeptide linker. In some embodiments, the CU can be a cell, and the SBE can be covalently attached to an antigen present on the surface of the cell. For example, the SBE can be a polypeptide covalently attached to a surface receptor of the cell. In some embodiments, the CU can be a solid support, and the SBE can be covalently attached to the surface of the solid support. For example, the SBE (e.g., a polypeptide SBE) can be covalently attached to the surface of a bead (e.g., a functionalized bead).

In some embodiments, a SBE can be non-covalently attached to a surface of the CU. In some embodiments, the SBE can comprise a binding moiety that can bind to a surface of the CU. In some embodiments, the CU can be a cell, and the SBE can comprise a binding moiety that can bind to an antigen present on the surface of the cell. For example, the SBE can comprise a ligand that binds to a surface receptor of the cell. In some embodiments, the SBE binding moiety can be an antibody moiety. In some embodiments, the CU can be a solid support, and the SBE can comprise a binding moiety that can bind to a surface of the solid support. For example, the SBE (e.g., a polypeptide SBE) can comprise a binding moiety that can bind to a surface of a bead (e.g., a functionalized bead).

In some embodiments, SBEs can be displayed as a part of glycosylphosphatidylinositol (GPI) anchored fusion protein. The recombinant structure of the fusion protein can be optimized to promote interaction between objects and, in particular, between cellular CUs and target objects. In some embodiments, the fusion protein construct can include domains of common yeast flocculation proteins, e.g., S. cerevisiae Flo1, Flo5, Flo9, Flo10 and Flo11. In some embodiments, the domains can include putative GPI associated moieties. In some embodiments, the domains can also include truncated fragments of the extracellular domains to increase the area of contact between adjoining objects, thereby increasing the strength of association between objects displaying complementary SBEs.

In some embodiments, a SBE can bind to a cognate binding partner. In some embodiments, the cognate binding partner can be a signal object (SO). In some embodiments, the cognate binding partner can be another SBE. In some embodiments, binding of the SBE to the other SBE can modulate an activity of the CU. In some embodiments, binding of the SBE to the other SBE can modulate the ability of the CU to produce an SO. In some embodiments, binding of the SBE to the other SBE can modulate the ability of the CU to degrade an SO. In some embodiments, the SBE can comprises an SO. In some embodiments, binding of the SBE to the SO can modulate an activity of the CU. In some embodiments, binding of the SBE to the SO can modulate the ability of the CU to produce an SO. In some embodiments, binding of the SBE to the SO can modulate the ability of the CU to degrade an SO.

In some embodiments, a SBE can be covalently attached to the surface of a sample object. In some embodiments, a SBE can be non-covalently attached to the surface of a sample object.

Engineered display of SBEs in various cellular systems has been disclosed in a number of publications, addressing a wide range of organisms ranging from phage and E. coli to yeast and general eukaryotic systems and also addressing protein folding, secretion, surface capture, and anchoring or tethering mechanisms.

In some embodiments, an SBE can be a cellular receptor. In some embodiments, receptor SBEs can be localized to the cellular membrane or cytoplasm. In some embodiments, receptor SBEs can include a transcription factor or a transmembrane receptor. In some embodiments, a SBE can be located on the surface of a cellular CU with a signaling moiety located on the cytoplasmic side of the cellular membrane. A cytoplasmic receptor can be any SO binding entity that can directly or indirectly lead to metabolic modulation, such as, but not limited to, a transcription factor or an aptamer that can change the transcription or translation rate of one or more genes. In some embodiments, a SBE can be a surface receptor or a transmembrane receptor protein. In cases where the CU can be derived from a yeast cell, a SBE can be a yeast receptor or its derivative (e.g., the signaling moiety of a yeast receptor fused to an engineered segment). Alternatively, a SBE can include a heterologous signaling moiety fused to a yeast signaling moiety and can incorporate other modifications for improved activity. In such a way, the SBE can incorporate homologous and/or heterologous segments of G protein-coupled receptors (GPCRs) in yeast derived CUs. Incorporated GPCRs can include modifications (e.g., compositions of extracellular, transmembrane, and cytoplasmic domains of GPCRs from different organisms and mutations improving their signaling properties) that can alter the receptors' specificities, sensitivities, and signaling activities. Incorporated GPCRs can also include modifications (e.g., mutations or truncations in cytoplasmic domains) that can alter post-translational regulation of receptor activity (e.g., degradation, molecular interaction). Non-limiting exemplary yeast GPCRs can include S. cerevisiae pheromone receptors STE2 and STE3 or their derivatives (e.g., mutants with altered stability). Non-limiting exemplary bacterial pheromone transcription factor proteins can include LuxR proteins that can sense bacterial pheromones N-acyl homoserine lactones.

### Internal States

In some embodiments, a CU can store information regarding past interactions with objects and external influences in its internal state. For the purposes of the present disclosure, the internal state of the CU can be not necessarily identical to the full state of the physical object as is defined by dynamical systems theory. Instead, the internal state contains information necessary to support and execute future computing actions. The internal state can include continuous variables (e.g., ionic concentrations, permittivities, permeabilities, internal pressures, absorbances, rigidities), discrete variables (e.g., entity copy numbers, degrees of polymerization, set of molecular conformations, molecular modifications), as well as spatial distributions (e.g., compartmentalization of entities, polarization). In some cases, for the purposes of computing system modeling, it can be convenient to define the state through probability distributions.

Internal states can be defined for both molecular and cellular CUs. Internal states of cellular CUs can be aptly described by biochemical reaction network models. Physical manifestations of the states, as related to the present disclosure, can be copy numbers of certain molecular species, in particular regulatory molecular species (e.g., transcription factors, regulatory RNAs, transferases) that contribute to maintaining cellular homeostasis. In some embodiments, the physical manifestations of the state can include copy numbers of active or inactive transcription factors in select cellular compartments. Wildtype transcription factors or their regulators can be considered. Heterologous transcription factors modified for the given host organism can also be considered. In addition, novel transcription factors can be considered. The internal state of the molecular CU can be set at time of production or through later interaction with objects or CUs.

### State Changes and Processing

In some embodiments, a CU can implement mechanisms that change its state following signal recognition or external influence. The effect of a mechanism can be predicted precisely (e.g., rapid and stable conformational change) or can have a stochastic nature (e.g., a change in an entity's time averaged copy number). In some embodiments, the state change can happen immediately following interaction of the SBE with its cognate ligand or following external influence. For example, the SO can interact directly with a transcription factor, or the transcription factor can undergo conformational changes as a result of a shift in e.g., temperature or illumination. In some embodiments, the state change can follow a transient internal process during which the state change can permeate but the effector process can be reset upon signal removal. The effector process can include one or more intermediate steps, wherein molecular species can undergo modifications that can build in parallel or in sequence. In some embodiments, the effectors can form a cascade, where the first effector can modify the second effector, and so on. In some embodiments, the cascade can also involve additional elements that can support or inhibit its progress. For example, eukaryotic cells can implement widely conserved MAPK cascades that make possible various signal processing functions and accept multiple regulators by which the cascades can be redirected and repurposed. In such example, the effector process can include an MAPK cascade coupled to adapter proteins and G proteins to signal sensing GPCRs.

In some embodiments, transient modification of e.g., transcription factor complexes following signal recognition can also serve in changing the internal state of a cellular CU. In some embodiments, a transcription factor can be modified directly by the SO. In some embodiments, a transcription factor can be modified as part of the transient process that can ensue following a signal recognition event. In some embodiments, activated transcription factors or associated elements can yield changes in transcription that can subsequently alter many other processes. In some embodiments, novel transcription factors can be used to transform signal recognition events into transcriptional changes. In some embodiments, novel transcription factors can allow pathway rerouting to promoters that can be independent of a native response. In addition, novel transcription factors can enable further modulation or signal processing.

In some embodiments, a CU can implement mechanisms that can process and change the state without external influence. In some embodiments, the mechanisms can stabilize the current state by nullifying the effects of random and external actions (e.g., regulation by negative feedback) or execute conditional state transitions that persist (e.g., periodic changes) or terminate in a finite number of steps (e.g., evaluation of logical operations).

In some embodiments, the internal state of the CU can affect intracellular entities that are not themselves part of the state. For example, in cases where the state determines gene activities, the current state of the system can determine the copy numbers of the corresponding gene products and thereby the states of any entity those products can affect. The affected entities can include SBEs. Hence, in some embodiments, the SBE profile of a CU can change as a result of a state change. The affected entities can include SBEs and any elements that can relay signal recognition events to other parts of the cell. The affected entities can include produced CUs or signal objects. In some embodiments, any of the entities affected through state change can be equally affected by signal recognition events or external influences.

In some embodiments, entities can be introduced that are not affected by signal recognition events, external influences, or internal state changes. These entities can be taken from the same family of entities as the reporter entities (i.e., fluorescent proteins, luminescent proteins, enzymes, etc.) and can be used to generate control measurements to which other measurements can be compared.

In some embodiments, the CU state change can detect cell state changes, such as, but are not limited to, cell specific apoptosis, cell specific activation, cell specific suppression, or stem cell differentiation. In some embodiments, the cell state change is cell specific apoptosis. In some embodiments, the Apoptosis Reaction Tubes comprise Reaction Reagent. In some embodiments, the Apoptosis Reaction Tubes comprises strain Annexin, strain EGFR, and strain BAR1 (SEQ ID NOs: 1-3). Further description of apoptosis detection is described in Examples 9-11.

### Reaction Medium

The Reaction Medium is a composition comprising a semi-permeable nutritive medium. In some embodiments, a material property of the Reaction Medium can be an optical property, an electrical property, or a thermal property. In some embodiments, the Reaction Medium has thermoresponsive properties.

The components of the Reaction Medium can be configured such that one or more computational clusters can be formed upon coming in contact with the at least one sample object, wherein each computational cluster comprises, independently, the at least one CUs.

In some embodiments, any components of the Reaction Medium described herein can be formulated with acceptable excipients, such as carriers, solvents, stabilizers, diluents, etc., depending upon a customized combination of CUs and target profile. Suitable excipients can include, for example, carrier molecules that include large, slowly metabolized macromolecules such as proteins, polysaccharides, polylactic acids, polyglycolic acids, polymeric amino acids, amino acid copolymers, and inactive viral particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like. In some embodiments, the Reaction Medium can contain hydrogel. In some embodiments, the hydrogel can be gelatin and/or sodium alginate. In some embodiments, the Reaction Medium can also contain yeast extract, peptone, D-glucose and/or an antibiotic (e.g., tetracycline, doxycycline, ampicillin, etc.). For example, the Reaction Medium can comprise gelatin, alginic acid sodium salt, yeast extract, peptone, D-glucose, and/or an antibiotic. In another example, the Reaction Medium can comprise gelatin, alginic acid sodium salt, yeast extract, peptone, D-glucose, and ampicillin.

In some embodiments, the Reaction Medium can be stored up to about 1 weeks, up to about 2 weeks, up to about 3 weeks, up to about 4 weeks, up to about 5 weeks, up to about 6 weeks, up to about 7 weeks, up to about 8 weeks, up to about 9 weeks, or up to about 10 weeks at room temperature without loss of signal. In some embodiments, the Reaction Medium can be stored up to about 2 weeks at room temperature without loss of signal. In some embodiments, the Reaction Medium can be stored up to about 1 weeks, up to about 2 weeks, up to about 3 weeks, up to about 4 weeks, up to about 5 weeks, up to about 6 weeks, up to about 7 weeks, up to about 8 weeks, up to about 9 weeks, up to about 10 weeks, up to about 11 weeks, up to about 12 weeks, up to about 13 weeks, up to about 14 weeks, up to about 15 weeks, or up to about 16 weeks at 4° C. without loss of signal. In some embodiments, the Reaction Medium can be stored up to about 8 weeks at 4° C. without loss of signal.

### Readout Reagent

The readout reagent is a composition comprising a reporter entity. In some embodiments, the readout reagent further comprises a buffer. A reporter entity can be any suitable molecular entity that can affect quantitative or qualitative measurements. Non-limiting exemplary reporter entities can include fluorescent proteins (e.g., GFP, RFP, YFP, or CFP), luminescent proteins (e.g., luciferase, such as, but are not limited to, Gaussia princeps luciferase (GLuc), Metridia longa luciferase (MLuc), Renilla renformis luciferase (RLuc), Cypridina noctiluca luciferase (CLuc)), enzymes (e.g., beta-lactamase, beta-galactosidase, SEAP), any functional fragments or variants thereof. The origin of these reporters and the coding sequences are fully disclosed in the current state of the art.

In some embodiments, signal recognition events, external influences, or state changes in the presently disclosed biocytometry system can lead to production of reporter entities. In some embodiments, the produced reporter entities can be cytoplasmic. In some embodiments, the produced reporter entities can be secreted and linked to the surface. In some embodiments, the produced reporter entities can be secreted and released into the medium of the system described herein. In some embodiments, secretion of reporter entities can increase their accessibility or increase their reporting function.

In some embodiments, entities can be introduced that are not affected by signal recognition events, external influences, or internal state changes. These entities can be taken from the same family of entities as the reporter entities (i.e., fluorescent proteins, luminescent proteins, enzymes, etc.) and can be used to generate control measurements to which other measurements are compared.

In some embodiments, a readout reagent comprises a luminescence substrate and a luminescence buffer. In some embodiments, the luminescence substrate and luminescence buffer can be mixed at 1:10 prior to assay readout. In some embodiments, the luminescence substrate and luminescence buffer can be mixed at 1:20 prior to assay readout. In some embodiments, the luminescence substrate and luminescence buffer can be mixed at 1:30 prior to assay readout. In some embodiments, the luminescence substrate and luminescence buffer can be mixed at 1:40 prior to assay readout. In some embodiments, the luminescence substrate and luminescence buffer can be mixed at 1:50 prior to assay readout. In some embodiments, the luminescence substrate and luminescence buffer can be mixed at 1:60 prior to assay readout. In some embodiments, the luminescence substrate and luminescence buffer can be mixed at 1:70 prior to assay readout. In some embodiments, the luminescence substrate and luminescence buffer can be mixed at 1:80 prior to assay readout. In some embodiments, the luminescence substrate and luminescence buffer can be mixed at 1:90 prior to assay readout. In some embodiments, the luminescence substrate and luminescence buffer can be mixed at 1:100 prior to assay readout.

### Signal Objects (SOs)

Signal objects (SOs), unlike CUs which are intended to generate new information, do not generate new information. SOs are intended to complement CUs by transferring information between entities. For practical purposes, a SO can be any object that can interact with an SBE. In some cases, an object can be both a SO and a CU. For instance, a SO can be fused to a molecular entity that itself has signal processing functions. This can result in a SO that can interact with other SOs or CUs that can be recognized by SBEs. In other cases, SOs can be suspended in the medium before association with their respective SBEs. In some embodiments, the SO can be produced by a CU in the system. In some embodiments, the SO can be degraded by a CU in the system. In some embodiments, the SO can be produced by a sample object in the system. In some embodiments, SOs can be added to a medium. In some embodiments, SOs can be generated by system entities (e.g., CUs and/or sample objects). In some embodiments, SOs can be generated in the medium from other SOs. In some embodiments, SOs can be generated within CUs from elementary metabolic precursors (e.g., by protein expression). In some embodiments, SOs can be secreted.

In some embodiments, secreted SOs can belong to families of yeast pheromones. Expression of yeast pheromones can then rely on the presence of either wildtype coding sequences or their recombinant derivatives introduced in tandem with appropriate regulatory sequences (e.g., promoters, untranslated regions, transcription factor binding sites). In some embodiments, yeast pheromones can belong to a family of lipidated pheromones of alpha factor type that can be expressed as precursors and secreted using non-traditional pathways. In some embodiments, yeast pheromones can belong to a family of peptide pheromones of the alpha-factor type that can be also expressed as precursors but can be secreted using traditional pathways. In some embodiments, several modes of regulation can be used to control and activate biogenesis of both pheromone types.

Suspended SOs can have various properties that can affect their recognition. In some embodiments, a SO can be membrane permeable and hence free to interact with cytoplasmic SBEs. In some embodiments, a SO can be membrane impermeable and hence can require SBEs that are exposed to the medium. In some embodiments, recognition of SOs can be inhibited by other mechanisms (e.g., hydrophobic sequestration) and can require additional treatments.

In some embodiments, SOs can be metabolites (e.g., amino acids, carbohydrates, ions, etc.), antibiotics (e.g., tetracycline, doxycycline, ampicillin, etc.), and various synthetic compounds (e.g., isopropyl beta-D-1-thiogalactopyranoside-IPTG, nitrocefin, anhydrotetracycline-aTc, toxins, etc.). In some embodiments, SOs can be biogenerated entities, e.g., signaling molecules of viral, bacterial, or mammalian origin. Well-known families of biogenerated signaling molecules can include, but are not limited to, bacterial acetylated homoserine lactones (AHL) and type 2 autoinducers, yeast mating pheromones, plant hormones, animal morphogens, and a wide array of mammalian hormones, cytokines, interleukins, chemokines etc. Biogenerated signaling molecules can also be engineered for altered specificity or function.

### Internal Signals

SOs produced by CUs can be referred to as internal signals. In some embodiments, an SO can be an internal SO.

In some embodiments, internal signals can be produced from other signals, e.g., by cleavage of existing signals or from metabolic precursors within cellular CUs. In some embodiments, internal signals can be engineered for altered behavior. In some embodiments, SOs or enzymes contributing to their productions can be expressed from recombined genes that comprise specific regulatory sequences (e.g., promoters, operators, untranslated regions). In some embodiments, signal behavior can be engineered through addition of synthetic open reading frame (ORF) elements. In some embodiments, single codon changes can redirect the signal to non-native SBEs.

In some embodiments, peptide signal degradation can be modulated by extending the terminal domains with a peptidase recognition tag. In some embodiments, peptide signals can be translated as pre-pro-peptides, where a series of additional cytoplasmic, periplasmic, or extracellular processing steps are required to produce mature pheromones. The pre-pro-peptide format can provide a platform for engineering signal activation, strength, and specificity and can therefore increase informational content of a single internal SO. In some embodiments, a SO can have multiple activity states, each of which can be characterized by its affinity for SBEs. In some embodiments, cleavage of pro-peptide sequences can transition SOs between the multiple activity states. In some embodiments, putative protease recognition sites within the pro-peptide sequence can be used to encode transitions between the multiple activity states.

In some embodiments, the peptide signals can belong to or be derived from a family of lipidated or non-lipidated yeast pheromones (e.g., S. cerevisiae alpha mating factor). For example, yeast pheromone pro-peptide sequences can be engineered to increase or decrease the signal strength of a single pre-pro-peptide by varying the number of mature peptides encoded within a single ORF. For example, pheromone coding sequences can be flanked by protease recognition sites and repeated within a single ORF. In some embodiments, the yeast pheromone alpha factor can be translated as a pre-pro-peptide with up to about four mature peptide coding sequences, the number of which can be increased or decreased using the same flanking motifs established in the wildtype sequence. In some embodiments, the pro-peptide sequence can also be extended to increase the number of activity states. In some embodiments, additional sequences that can include recognition sites for non-native proteases can be inserted into wildtype sequence. By such modification, intermediate activity states can be produced, wherein the SOs recognizes SBEs that are different from those recognized by the fully mature pheromone.

In some embodiments, the mature pheromone coding sequence can also be altered. In some embodiments, mature pheromone sequences from one species can be exchanged for pheromone sequences from another species. Previous work has shown that crosstalk between pheromone-receptor pairs from different species can be negligible. In some embodiments, internal signals can also include signaling molecules from unrelated species. Such internal signals can exhibit properties desirable for some applications.

In some embodiments, internal signals can be membrane permeable and hence detectable by simple mechanisms. In some embodiments, internal signals can provide complete orthogonality with respect to other signal objects in a medium (e.g., plant hormones). In some embodiments, SOs can be targeted towards SBEs of target objects in a medium. Such signals can include mammalian cytokines, chemokines, interleukins, growth hormones, neuropeptides, etc.

In some embodiments, recombination technologies can be used to produce heterologous signals in various cellular CUs. In some embodiments, yeast cells can provide a cellular platform wherein signals of various origins (e.g., bacterial, mammalian, or other higher eukaryotes) can be produced. In some embodiments, production of such signals can require additional metabolic engineering to enable necessary post-translational modifications and metabolic processing.

In some embodiments, internal signals can also exhibit properties that can enable their easy measurement by external devices. Measurable signals can include molecules with high specificity that can be quantified directly (e.g., immunosorbent assay or polymerase chain reaction). Measurable signals can also include molecules that can alter bulk properties (e.g., absorbance, fluorescence, etc.) of a medium comprising the presently described system. In some embodiments, easily measurable internal signal can primarily be used for readout of device states. Non-limiting exemplary internal signals can include fluorescent proteins (e.g., GFP, RFP, YFP, or CFP), luminescent proteins (e.g., luciferase, such as, but are not limited to, Gaussia princeps luciferase (GLuc), Metridia longa luciferase (MLuc), Renilla renformis luciferase (RLuc), Cypridina noctiluca luciferase (CLuc)), enzymes (e.g., beta-lactamase, beta-galactosidase, SEAP), any functional fragments or variants thereof.

The internal signal classification does not bar a signal from also being classified as external. For instance, signals that are produced within a medium comprising the presently described system, isolated, and manually returned to the medium at a later time can be both internal and external signals.

### External Signals

SOs that are not produced by CUs can be referred to as external signals. In some embodiments, an SO can be an external SO.

In some embodiments, external signals can include synthesized molecules or purified biogenerated products. In some embodiments, external signal can include metabolites and related compounds or short peptides. In some embodiments, SOs can be added to a medium comprising the presently described system, and the added SOs can include transcriptional inducers (e.g., IPTG, aTc, AHL), classes of amino acids (e.g., aromatic amino acids), pheromones (e.g., alpha factor), etc.

In some embodiments, external signals can include optical signals that can illuminate a medium comprising the presently described system. In some embodiments, external signals can include magnetic signals that can magnetize system objects. In some embodiments, external signals can include electrical signals that can polarize object charge.

In some embodiments, external signals can also be produced by entities in the input sample. In some embodiments, such signals can include a wide array of molecules that can be well-mixed throughout a medium comprising the presently described system. In some embodiments, such signals can be localized to specific target objects. Well-mixed signals can serve to coordinate system wide computing functions. Localized signals can affect only those CUs that recognize the target object (i.e., CUs with SBEs that interact with SBEs displayed by the target object class).

Production of external signals can be constant in time or time-varying according to some predetermined pattern (e.g., exponentially decaying in time).

### Output Signals

In any of the methods described herein, the logical operator modules can receive one or more input signals (e.g., sample objects) and generate one or more output signals (e.g., output SOs) based on the specified modules.

### Signal Modulations

In some embodiments, a CU can affect internal SOs or external SOs. A CU can affect a single or multiple signals directly (e.g., by producing, degrading, or transforming signals) or indirectly (e.g., by producing CUs). In some embodiments, effects on SOs can be constant or time-dependent, where changes can occur spontaneously, following a change in internal state, or during signal induction. Signals can be affected while at least partially exposed to the medium of the present disclosure (i.e., Reaction Reagent+Reaction Medium). In some embodiments, a CU can catalyze processing of signals. Such a catalyst can be an enzyme that can nullify SOs by affecting their degradation or an enzyme that can change the specificity of the SO towards SBEs. For example, an inactive SO can be split into one or more active SOs recognized by specific SBEs. For example, in the case of peptide signals or their derivatives, the enzyme can be a protease that can specifically or nonspecifically recognize and cleave the signal amino acid sequence. In some embodiments, purified proteases can be added to the medium of the present disclosure (i.e., Reaction Reagent+Reaction Medium) in an active or inactive form that can be cis or trans activated. A wide variety of commercially available proteases can be used for this purpose. In some embodiments, heterologous, native, or engineered proteases can also be secreted by the CUs.

In some embodiments, CUs can affect signals directly by secreting SOs into the medium. In some embodiments, SOs and cellular CUs can be secreted and linked to the CU surface. In some embodiments, SOs and cellular CUs can be secreted and released into the medium. Such secretion can be constant in time, induced by recognized signals, or induced following an internal state change. In some embodiments, intracellular SOs can be loaded in the CU any time prior to use (e.g., by electroporation or other disclosed methods for peptide transfection); however, in most cases, intracellular SOs can be synthesized by the CUs using available or engineered metabolic processes. In some embodiments, SOs can be produced from available metabolites by appropriate synthesizing enzymes. In some embodiments, polypeptide SOs can be produced by either heterologous or homologous gene expression, where the expression itself can be either constant in time or time-dependent with changes occurring either upon signal induction or internal state change. In some embodiments, SOs or potential CUs can be stored by the current CU for a period of time prior to secretion or secreted directly. Storage of SOs and their conditional secretion can be accomplished by synthesis of object precursors and rapid secretion following final processing (e.g., addition of functional groups or release by cleavage of pre-domains), where the processing itself can be induced through regulated gene expression or post-translational activation of catalyzing agents.

In some embodiments, biogeneration of SOs can require heterologous metabolic mechanisms. In such case, the secreted SOs can be recognized by entities in the input sample, rather than being recognized by other CUs. In such cases, the CU can be metabolically engineered to produce and regulate the various metabolic factors that can be necessary to produce the SOs from the native metabolic precursors.

In some embodiments, CUs can affect signals indirectly by producing CUs of the same or different type. In some embodiments, the capability of CUs to produce or degrade SOs can be enhanced or attenuated by binding of the SBE to its cognate binding partner. In some embodiments, the cognate binding partner can be a SBE associated with at least one CU. In some embodiments, the cognate binding partner can be a SBE associated with at least one sample object. In some embodiments, the cognate binding partner can be another SO.

In some embodiments, a sample object can be associated with an SBE and can produce or degrade an SO. In such embodiment, the capability of the sample object to produce the SO can be enhanced or attenuated by binding of the SBE to its cognate binding partner. In some embodiments, the cognate binding partner can be a SBE associated with at least one CU. In some embodiments, the cognate binding partner can be a SBE associated with at least one sample object. In some embodiments, the cognate binding partner can be another SO.

### Signal Recognition

In some embodiments, a CU can be affected by internal or external SOs through interaction with an SBE. In some embodiments, a SBE can be specific for a single signal or multi-specific for a subset of signals with variable sensitivity for each member of the subset. In some embodiments, a SBE can recognize a signal through physical interactions, e.g., hydrogen bonds, van der Waals forces, hydrophobic interactions, and/or ionic interactions. In some embodiments, a SBE can display some change in activity once an interaction can be initiated. In some embodiments, signal interactions can activate or inhibit e.g., an enzymatic process of a molecular CU with a SBE moiety and an enzymatic function. In some embodiments, a CU can be a protease with a SBE located near its active site, where signal interactions can lead to protease inhibition. In some embodiments, signal interactions with a SBE does not necessarily prevent processing of the SO. For example, further processing of the signal interaction by another protease can reverse an inhibitory effect of the original signal.

In some embodiments, a SBE can be located on the surface of a cellular CU with a signaling moiety located on the cytoplasmic side of the cellular membrane. In some embodiments, a SBE can be a surface receptor or a transmembrane receptor protein. In some embodiments, a SBE can be a cytoplasmic protein with a binding moiety. In some embodiments, a SBE can be a transcription factor with a sensory domain that can recognize membrane permeable SOs.

In some embodiments, a CU can be configured to respond to an SO in the medium of the present disclosure (i.e., Reaction Reagent+Reaction Medium). In some embodiments, the CU can be configured to respond to an SO in the medium by enhancing the level of the SO in the medium. In some embodiments, the SO can be an internal SO that can be recognized by at least one CU. In some embodiments, the SO can be an internal SO that can be recognized by at least one sample object. In some embodiments, the SO can be an output SO, and the output signal can comprise the SO level. In some embodiments, a CU can produce an SO, and interaction of the SO with the CU can enhance the capability of the CU to produce the SO. In some embodiments, the CU can be configured to respond to the SO by producing another SO, wherein interaction of the other SO with another CU can enhance the capability of the other CU to produce the SO.

### Computational Clusters

According to any of the methods provided herein, the CUs can form one or more clusters, also referred to herein as a computational cluster, in a medium (i.e., Reaction Reagent+Reaction Medium) comprising the presently described system, upon coming in contact with at least one sample object derived from an input sample. In some embodiments, the computational cluster can further comprise at least one sample object derived from the input sample. In some embodiments, the computational cluster can comprise one or more CUs, and optionally one or more sample objects.

In some embodiment, the computational clusters can be formed after a sufficient amount of time in an incubator. In some embodiments, the sufficient amount of time can be about 2 hours, about 2.1 hours, about 2.2 hours, about 2.3 hours, about 2.4 hours, about 2.5 hours, about 2.6 hours, about 2.7 hours, about 2.8 hours, about 2.9 hours, about 3 hours, about 3.1 hours, about 3.2 hours, about 3.3 hours, about 3.4 hours, about 3.5 hours, about 3.6 hours, about 3.7 hours, about 3.8 hours, about 3.9 hours, about 4 hours, about 4.1 hours, about 4.2 hours, about 4.3 hours, about 4.4 hours, about 4.5 hours, about 4.6 hours, about 4.7 hours, about 4.8 hours, about 4.9 hours, about 5 hours, about 5.1 hours, about 5.2 hours, about 5.3 hours, about 5.4 hours, about 5.5 hours, about 5.6 hours, about 5.7 hours, about 5.8 hours, about 5.9 hours, or about 6 hours. In some embodiments, the sufficient amount of time can be about 3 hours. In some embodiments, the sufficient amount of time can be about 3.5 hours. In some embodiments, the sufficient amount of time can be about 4 hours. In some embodiments, the sufficient amount of time can be about 4.5 hours. In some embodiments, the sufficient amount of time can be about 5 hours.

In some embodiments, the computational cluster can be formed when the CUs can associate with sample objects by diffusion of the CUs and the sample object in a medium comprising the presently described system. In some embodiments, the computational cluster can be formed when a force can be applied to a medium comprising the presently described system, such that the CUs and the sample objects can be placed in close proximity. In some embodiments, the force can be a centrifugal force. In some embodiments, the centrifugal force can be continuous. In some embodiments, the continuous centrifugal force can be applied by twisting the reaction tube around its z-axis between spin-downs, wherein such twisting of the reaction tubes can create a sliding effect along the reaction tube wall, promoting better interactions between Sample Objects and CUs. In some embodiments, the force can be a magnetic force. In some embodiments, the force can be an electrostatic force. In some embodiments, the computational cluster can be formed when the CU can be configured to respond to an SO present in a boundary layer around the cluster by producing another SO that can be not restricted to the boundary layer.

In some embodiments, the computational cluster can comprise a first CU, and the presently described system can further comprise a second CU that can be not localized to the cluster, wherein the second CU can be configured to degrade an SO produced by the first CU.

### Logical Operator Modules

The CUs of the present disclosure can enable a rich set of behaviors. Standard mathematical notation can be introduced to precisely describe certain orchestrated functionalities of CU compositions. Functions, which can be made up in part with arithmetic operators and logical operators, can be encoded and evaluated by the CUs and signals that interact and self-organize within the medium of the present disclosure (i.e., Reaction Reagent+Reaction Medium). The input that drives the computation can be the sample objects derived from an input sample.

From the perspective of computing theory, the computation of the present disclosure can implement a rich set of operations that can include all forms of combinational logic and sequential logic. In other words, the fundamental configurations provided below can be the building blocks that can form a functionally complete system of Boolean operations. Moreover, the internal states of the CUs jointly make up the composite system state that can condition future Boolean operations for sequential logic functions.

The presently described systems and the methods of using the systems utilize Boolean logical operations. The logical operator modules of the present disclosure can act as logical circuits that can perform logical operations. A non-limiting exemplary logical operator module can receive one or more input signals (e.g., sample objects) and can generate one or more output signals (e.g., output SOs). In some embodiments, a logical operator module can comprise one or more CUs. In some embodiments, a logical operator module can comprise at least one sample object and at least one CU. In some embodiments, a logical operator module can comprise at least one sample object and two or more CUs. In some embodiments, a logical operator module can generate one or more output signals. In some embodiments, the methods of the present disclosure can comprise at least one logical operator modules. In some embodiments, a logical operator module can comprise a YES gate, an AND gate, a NAND gate, an OR gate, a NOR gate, a XOR gate, a XNOR gate, a NOT gate, or any combination thereof.

In some embodiments, a logical operator module can operate as a YES gate, wherein the YES gate can comprise generating one or more output signals only when both a first CU and a second CU can interact with at least one sample object.

In some embodiments, a logical operator module can operate as an AND gate, wherein the AND gate can comprise generating one or more output signals only when both a first CU and a second CU can interact with at least one sample object.

In some embodiments, a logical operator module can operate as a NAND gate, wherein the NAND gate can comprise suppressing or diminishing one or more output signals when both a first CU and a second CU can interact with at least one sample object.

In some embodiments, a logical operator module can operate as an OR gate, wherein the OR gate can comprise generating one or more output signals when either a first CU or a second CU or when both the first CU and the second CU can interact with at least one sample object.

In some embodiments, a logical operator module can operate as a NOR gate, wherein the NOR gate can comprise generating one or more output signals when both a first CU and a second CU are not interacting with at least one sample object.

In some embodiments, a logical operator module can operate as a XOR gate, wherein the XOR gate can comprise generating one or more output signals when either a first CU or a second CU but not both CUs can interact with at least one sample object.

In some embodiments, a logical operator module can operate as a XNOR gate, wherein the XNOR gate can comprise generating one or more output signals when either both a first CU and a second CU or when both the first CU and the second CU can interact with at least one sample object.

In some embodiments, a logical operator module can operate as a NOT gate, wherein the NOT gate can comprise suppressing or diminishing one or more output signals when a first CU can interact with at least one sample object.

Negation can be an essential aspect of any general Boolean network since it can allow for recognition of absent SBEs. Negated implication can be a negation of all outputs so that an output can be true only if first input can be true and second input can be false. To compute a negated implication, the configurations can be further extended with signal degrading objects. In some embodiments, signal degradation can be achieved by target associated CUs. This can comprise secretion of signal degrading enzymes or display of signal degrading enzymes by target associated CUs.

More detailed information on configurations and operations of the computation comprising the presently described entities are described in International Patent Application No. PCT/US2019/50068 and U.S. Patent Publication No. US 2021-0319279, each of which are incorporated by reference in its entireties for all purposes.

### Systems of the Disclosure

Provided herein is a system for biological computing of the presently disclosed methods, the system comprising: (a) at least one sample object derived from an input sample; (b) a Reaction Reagent comprising at least one computing unit (CU) configured to interact with the at least one sample object; (c) a Reaction Medium; and (d) a readout reagent comprising a reporter entity and a reporter buffer. In some embodiments, the system can further comprise one or more evaluation strips and a plate adopter. In some embodiments, the system can further comprise one or more 96-well plates and one or more microcentrifuge tubes. In some embodiments, the system can further comprise a thermocycler. In some embodiments, the system can further comprise a plate reader.

In some embodiments, reactions can be analyzed separately or in bulk on any plate reader with readout reagent (e.g., luminescence) functionality. In some embodiments, the plate reader with luminescence functionality has sensitivity ≤100 amol ATP. In some embodiments, the read setting of the plate reader with luminescence functionality can be endpoint/kinetic read type, luminescence fiber optics type, 255 gain, 1 s integration time, auto-adjust or maximum read height, custom (defined by user) plate type, and 5 reads (average used as the final readout values).

In some embodiments, the presently described systems can maintain single target sensitivity in the presence of high background. See Example 4. In some embodiments, the presently described systems can exhibit exceptional false positive rate characteristics. See Example 5.

The present disclosure implements a computing device. The presently disclosed biocytometry methods can be organized in a computing architecture that can cast broad terms from computer science into the device domain. Standard computer architectures (e.g., von Neumann or Harvard architectures) are well known and widely used to organize operations of stored-program electronic digital computers. More detailed information of the biological computing architecture and its implementation comprising the presently described entities are described in International Patent Application No. PCT/US2019/50068 and U.S. Patent Publication No. US2021-0319279, each of which are incorporated by reference in its entireties for all purposes.

### Object Clustering and Signal Evaluation

In some embodiments, a clustering reaction can be used to ensure SBE recognition can be completed for biological computing. In various embodiments, object sizes (e.g., cellular CUs) can be in the order of micrometers. At these scales, exogenous forces can be necessary to achieve sufficient mixing of objects. For example, a 1 μm particle can diffuse 1000 times slower than a 1 nm particle. The clustering reaction can use other mechanisms besides diffusion to promote interaction between SBEs. At the same time, the clustering reaction must minimize the effects that these mechanisms have on signaling. Forced interaction between objects can resemble object co-localization, which can be the basis for most computational operations. Hence, the clustering reaction must also implement mechanisms that prevent signaling between objects.

The clustering method of the present disclosure can simultaneously force interaction and block signaling. During the clustering reaction, system objects (i.e., sample objects and CUs) can be. Combination of objects can be done all at once or progressively. In some embodiments, the reaction can commence with resuspension of objects in the medium of the present disclosure, which can be optimized for specific clustering (e.g., low viscosity, high ion content, containing blocking agents or solubility agents). The medium can also contain signal blockers (e.g., binding compounds or signal degrading enzymes) or metabolic suppressors (e.g., translation inhibitors). Alternatively, the medium can be depleted of essential metabolites (e.g., amino acids, carbon sources) required for high levels of signal production. In some embodiments, the reaction can proceed by forcing object interaction (e.g., hydrodynamic mixing, electromagnetic manipulation, mechanical compression). The choice of mechanism can depend on object properties. Hydrodynamic mixing can be appropriate in mixtures comprising objects of various masses or size. Electromagnetic manipulation can be applicable in mixtures where target objects or CUs can be magnetizable. Objects can be magnetized by linkage (e.g., covalent or non-covalent bonding) to magnetizable entities or by loading (e.g., by electroporation, diffusion, carrier particles) the objects with magnetizable particles. Mechanical compression (e.g., by centrifugation) can require no special properties but can be more prone to erroneous processing. In all cases, the clustering reaction can be performed in a one-time batch process, continuously throughout the computing process (e.g., in a reactor), or repeated at multiple times during the computing process. In each execution, the results of the reaction can be clusters that can co-localize CUs.

In some embodiments, the signal production can be binary, that is, either a signal can be transmitted or signal can be not transmitted. In some embodiments, the signaling processes (e.g., signal production, signal degradation, signal recognition, state transition, etc.) can be mediated within a reaction optimized for signal evaluation. The evaluation reaction can initiate or terminate signal production and regulate chemical or spatial conditions. The primary goal can be to protect cluster integrity and to ensure completion of relevant physiological and enzymatic processes.

In some embodiments, the evaluation and clustering reactions can occur simultaneously. In some embodiments, the evaluation reaction can be explicitly initiated following the clustering reaction. In such cases, the two reactions can share the same medium but remain separate through changes in temperature, object density, or illumination. In some embodiments, separation of the two stages can include a medium exchange. Whereas the medium of the clustering reaction can be optimized for interaction (e.g., low viscosity, increased ion content, added blocking entities and solubility agents), the medium of the evaluation reaction can include enriched signaling precursors. The medium can be enriched in metabolites that can support signal production and signal recognition. For instance, synthetic growth media can be optimized for CU metabolisms and enriched with inducers of wild type or recombinant cellular systems. In addition, the properties of the media (e.g., pH, salt content) can be optimized for buffering and for any extracellular enzymatic processes (e.g., proteolysis, hydrolysis).

Spatial arrangement of the evaluation reaction can affect diffusion. Convective diffusion can weaken signal strength requiring more sensitive SBEs that can be more error prone. Hence, the evaluation reaction can take precautions to limit object motion. For instance, medium viscosity can be increased by addition of certain polysaccharides or other polymers. In addition, the additives can be cross-linked, forming a structured matrix that can trap and immobilize system objects. The result can be greater signal accumulation and decreased signal transmission between clusters.

In some embodiments, according to any of the systems described herein, the system can further comprise an agent that can increases the viscosity of a medium comprising the system. In some embodiments, the agent can be a polymer. In some embodiments, the polymer can be a polysaccharide. In some embodiments, the agent can be cross-linked to form a matrix configured to immobilize one or more of the system components.

### Kits of the Disclosure

The present disclosure provides a kit that contains any of the above described system components and compositions described herein. Provided herein is a kit for biological computing, the kit comprising: (a) one or more reaction tubes comprising Reaction Reagent; (b) one or more master tubes comprising Reaction Medium; (c) a readout reagent comprising a reporter entity and a reporter buffer; and (d) an instruction for use of the kit. In some embodiments, the kit can further comprise one or more evaluation strips and a plate adapter. In some embodiments, the kit can further comprise a blank sample.

In some embodiments, the blank sample can be prepared with the Reaction Medium and the Reaction Reagent of the present disclosure and can constitute a reaction tube with resuspension buffer used in a place of a test sample. In some embodiments, sample readout signal can be normalized using the following formula:

$$SNR = \frac{Signal - Blank}{Blank}$$

In some embodiments, the plate adapter can accommodate up to 12 evaluation strips at once. In some embodiments, prior to first use, the plate adapter can be defined as a standard 96-well plate with adjusted parameters of 22000 m plate height and 5000 m well diameter.

In some embodiments, quantity of Reaction Reagent in the reaction tube can determine the processing power of a reaction. In some embodiments, the active component of the presently described kit is supplied in dried form in the reaction tube.

In some embodiments, the kit can contain evaluation strips. In some embodiments, the evaluation strips can be a set of standard PCR strips. In some embodiments, up to 8 tubes can be used to analyze a reaction. In some embodiments, black and white PCR strips can be alternatively used, but read setting may need to be adjusted to account for the shift in signal intensity.

The instructions for practicing the presently described methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g., CD-ROM, diskette, flash drive, etc. In some instances, the actual instructions are not present in the kit, but means for obtaining the instructions from a remote source (e.g., via the Internet), can be provided. An example of this embodiment can be a kit that includes a web address where the instructions can be viewed and/or from which the instructions can be downloaded. As with the instructions, this means for obtaining the instructions can be recorded on a suitable substrate.

### EXAMPLES

These examples are provided for illustrative purposes only and not to limit the scope of the claims provided herein.

### Example 1. Engineering of the Biocytometry System

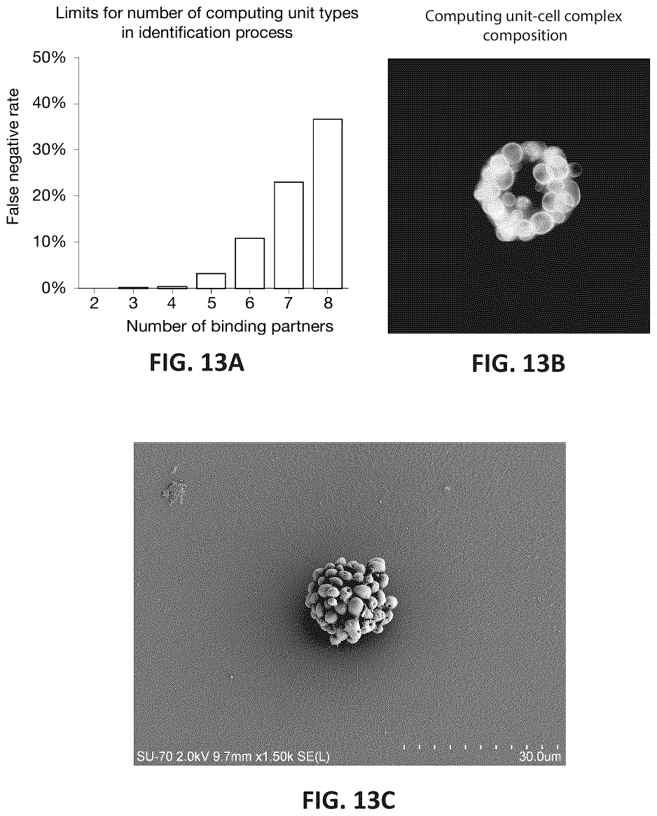

This Example describes development of the presently described biocytometry system for the identification of target cell immunophenotypes in suspension. The specificity towards targeted immunophenotypes was determined by the combination of computing unit types, where each was defined by distinct binding and signaling properties, and configurable for surface marker logical gating—AND, OR, YES. For example, a computing unit combination annotated as BIOS(EpCAM) operated under a YES gate to selectively identify EpCAM+ cells, while BIOS(EpCAM+EGFR) enabled identification of EpCAM+EGFR+ target cells. Up to 5 computing unit types and a total of 10 or more computing unit contributed to a single identification event (FIGS. 13A-13C).

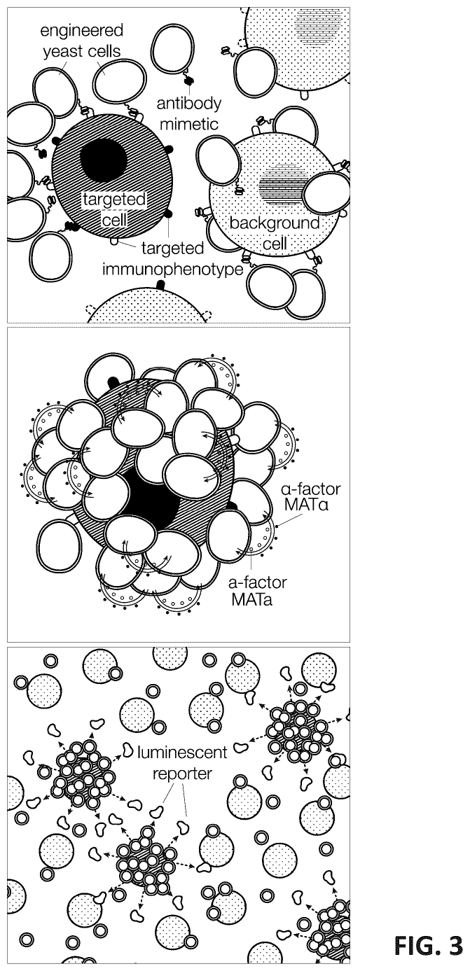

The computing units were based on an engineered strain of S. cerevisiae S288C, wherein (i) novel anchor system enabled efficient formation of computing unit-cell complexes (FIG. 3, top); (ii) hypersensitized signaling pathway facilitated robust inter-computing unit communication and accurate interpretation of cellular immunophenotypes (FIG. 3, center); (iii) a reporter module relayed the output of the logical processing to a luminescent reporter gene (FIG. 3, bottom); and (iv) integral to the biocytometry system was a semi-permeable hydrogel matrix that isolated inter-computing unit communication, permitting single-cell resolution in immunophenotype processing without sample compartmentalization.

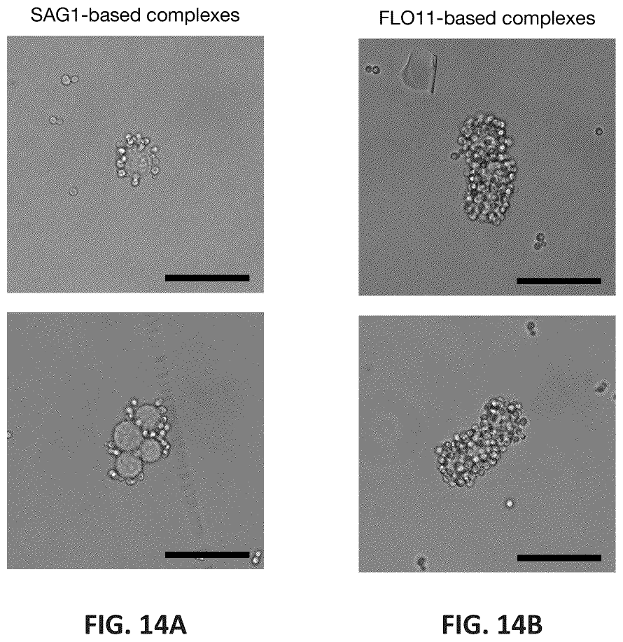

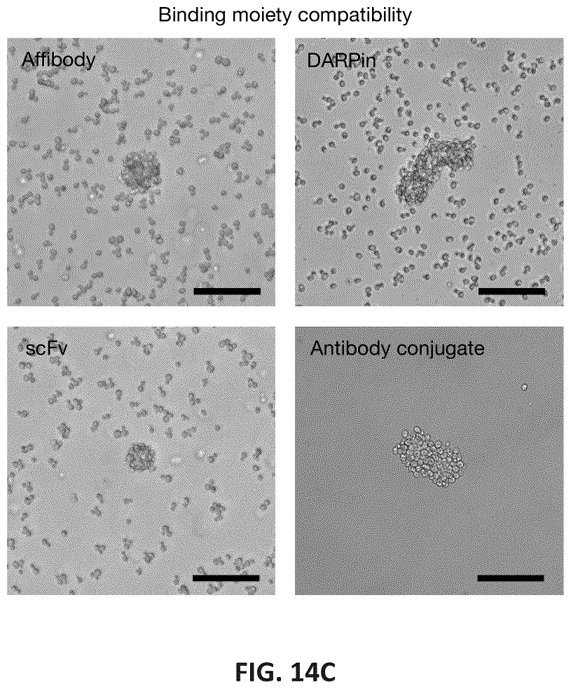

Antibody mimetics displayed on the surface of computing units enabled the formation of immunospecific computing unit-cell complexes. Anchor domains in existing yeast display systems, although extensively used for antibody engineering and protein-protein interaction studies, demonstrate limited efficacy in complex formation (FIG. 14A). A novel cell wall anchor domain was engineered that employed a truncated version of yeast FLO11, spanning from the central region to the GPI anchor (residues 206-1367). Comparative assessments with HaCaT cells revealed a 26.6-fold increase in binding efficiency for computing units using the FLO11 anchor system over the conventional SAG1 anchor (FIG. 14B). Notably, the presently described system enabled seamless integration of established proteinaceous binding moieties, including but not limited to antibody conjugates, scFvs, DARPins, and Affibodies (FIG. 14C).

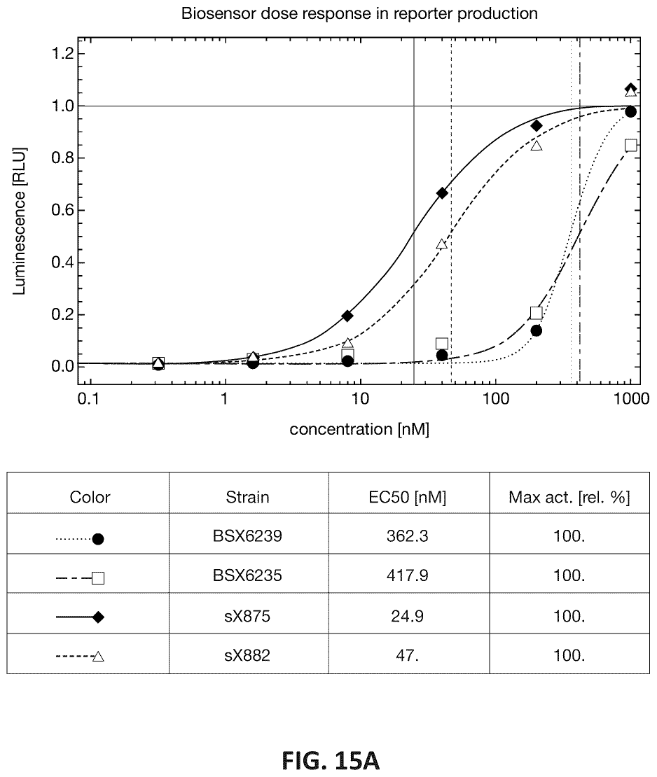

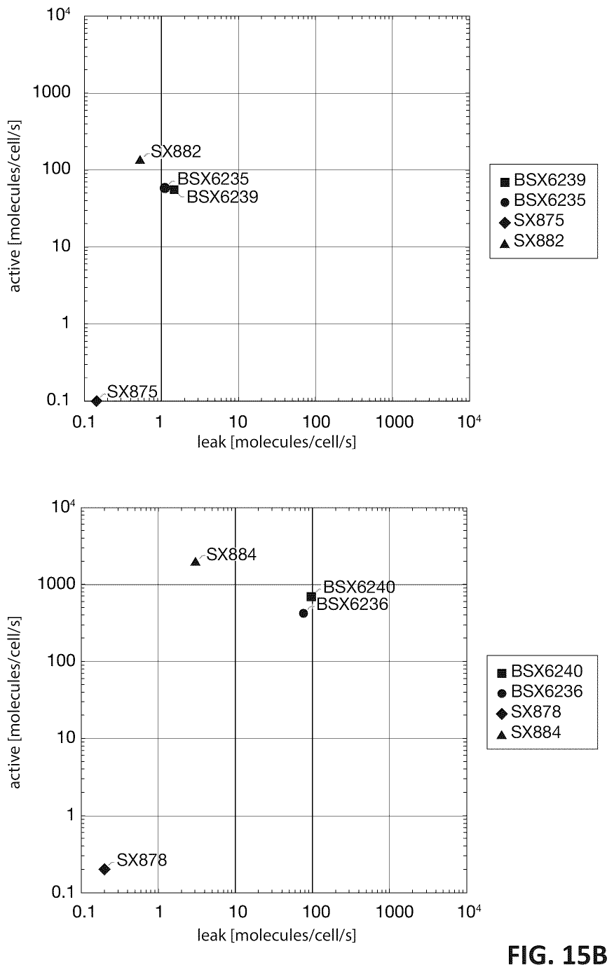

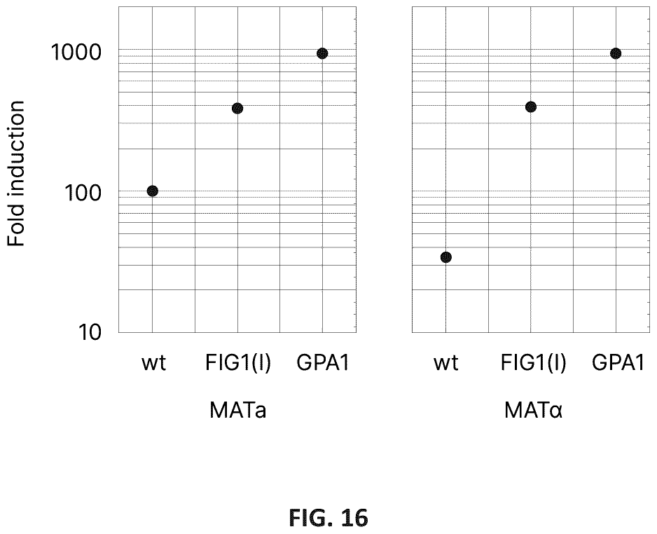

A re-directed mating pathway governs inter-computing unit communication and facilitates accurate immunophenotype interpretation of analyzed cells. Mating pathway activation in the computing unit is conditional on the adjacency of the cognate signaling partners in the cell-computing unit complex. Yeast computing units of two mating types, MATa and MATa, capable of producing and detecting mating factors (MFA1, MFX1) through GPCR receptors (STE2 and STE3), were utilized. The strains were engineered for enhanced mating factor sensitivity and tunable production through the implementation of mating-type-specific alterations. A significant enhancement in both mating pheromone sensitivity (4-fold and 23-fold for MATa-origin and MATa-origin strains, respectively) and mating pheromone production capacities (4-fold and 38-fold for MATa-origin and MATa-origin strains, respectively) was observed when compared to their wildtype parent strains (FIGS. 15A-15B).

The reporter system provides for a rapid and sensitive identification of activated computing units. Detection of a single activated computing unit-cell complex necessitates a system with exceptional induction fold change, high yield, and rapid expression kinetics. It was observed that NanoLuc luciferase significantly enhanced promoter fold-induction properties by reducing basal expression 5-fold and increasing maximal expression levels by 1.5-fold. A dampening effect on the basal activity of the mating pathway was achieved, leading to 242% increase in its fold-induction (FIG. 16). Collectively, these alterations yielded a 10-fold enhancement in reporter gene fold-induction relative to the wild-type strain using a FIG1p-regulated reporter (FIG. 16). The system's modular architecture permitted the integration of alternative reporters like eGFP23 or β-lactamase 24.

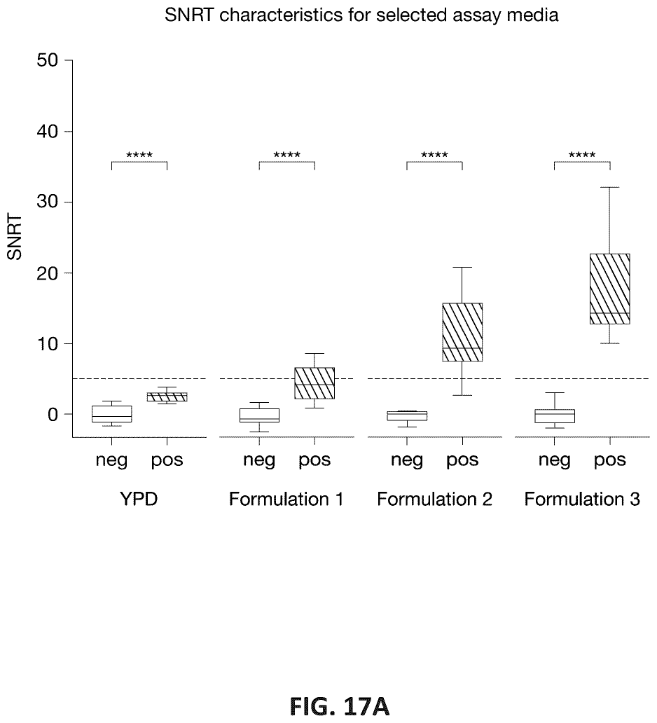

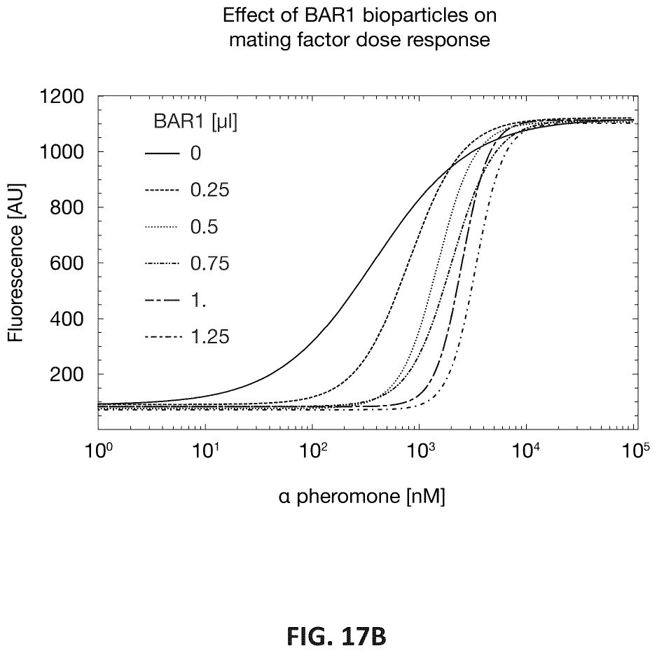

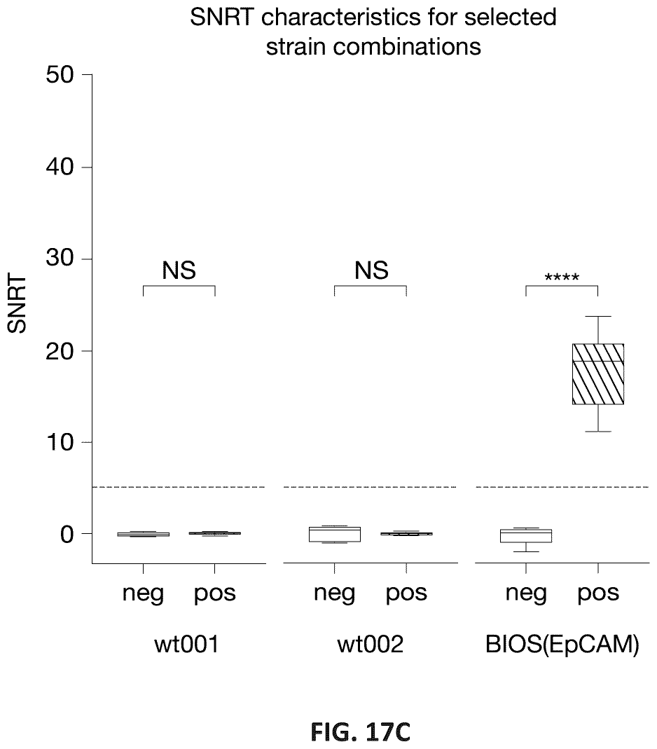

A functionalized semipermeable hydrogel matrix was crucial for confining communication to computing unit-cell complexes and minimizing intercomplex cross-talk. Conventional single-cell analysis frequently necessitates sample compartmentalization, utilizing either drop-based or spatiotemporal isolation techniques. To circumvent these limitations, biocytometry employs a hydrogel system formulated from a blend of biopolymers that enables free nutrient diffusion while accentuating local gradients of mating pheromones and actively suppressing their dispersion (FIG. 17A). Additionally, computing unit expressing BAR1 protease were incorporated into the hydrogel system, acting as concentration sinks to regulate the overall concentration of mating pheromones (FIG. 17B). The synergistic interplay between the hydrogel matrix and the hypersensitized signaling pathway was pivotal for the fundamental functionality of the biocytometry system (FIG. 17C).

### Example 2. Technical Validation with Cell Lines and Multiparametric Target Cell Identification

This experiment was performed to demonstrate the capabilities of the presently described biocytometry system.

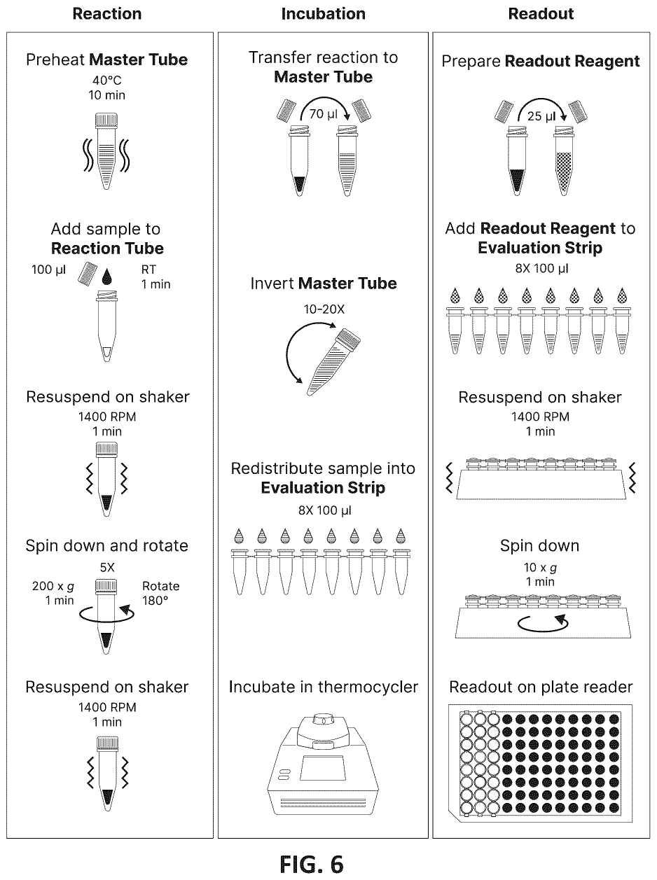

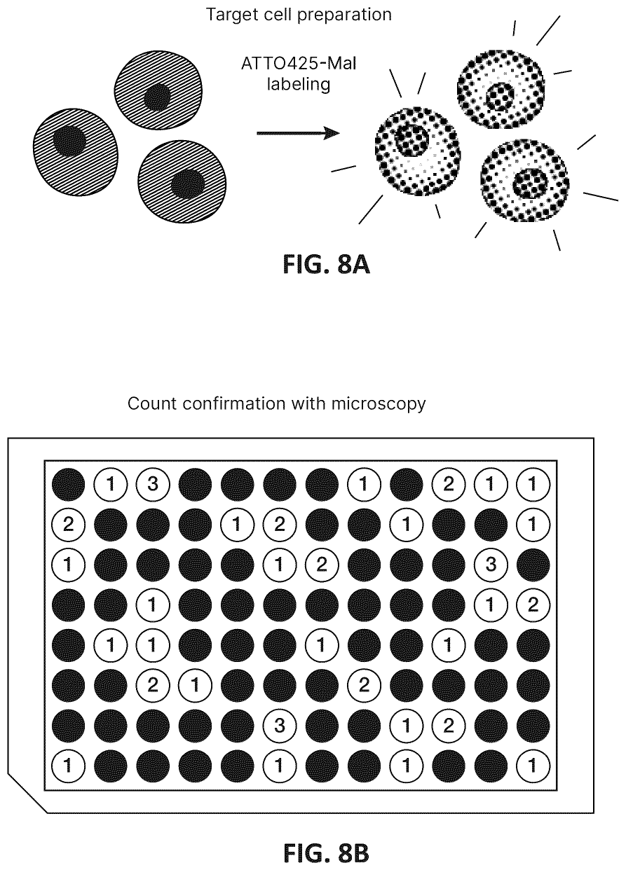

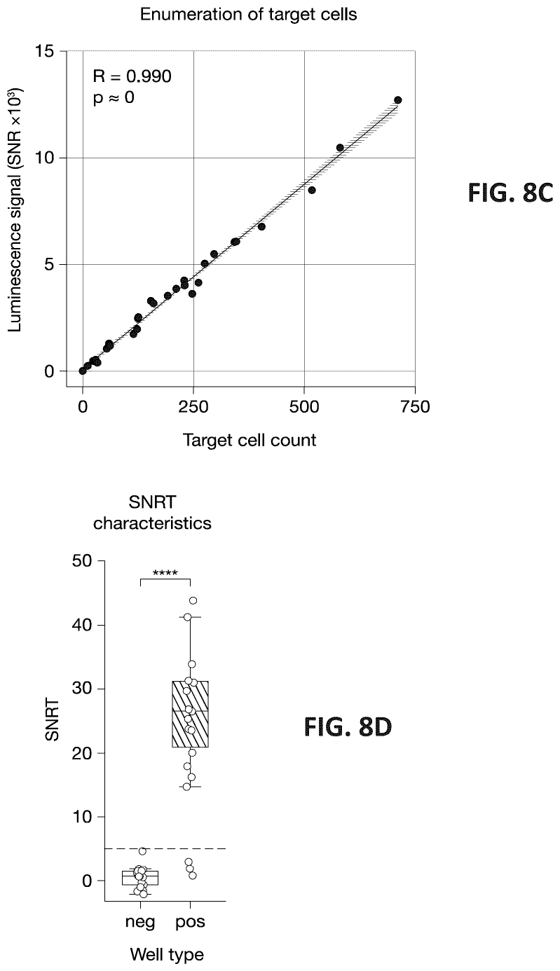

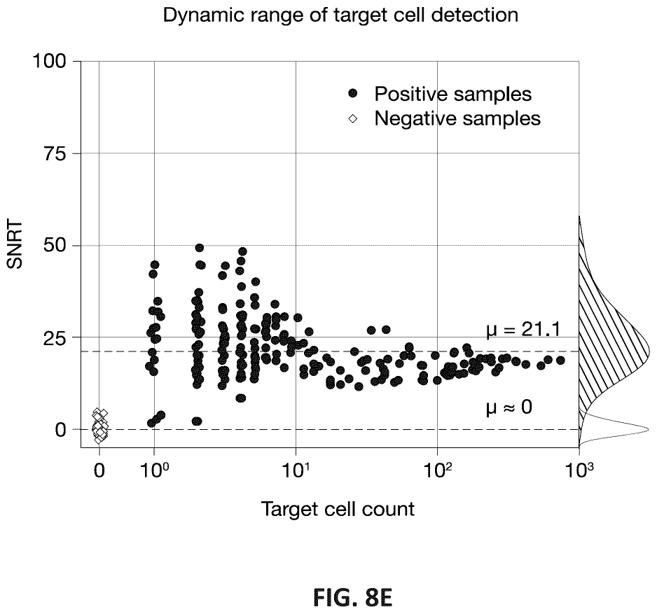

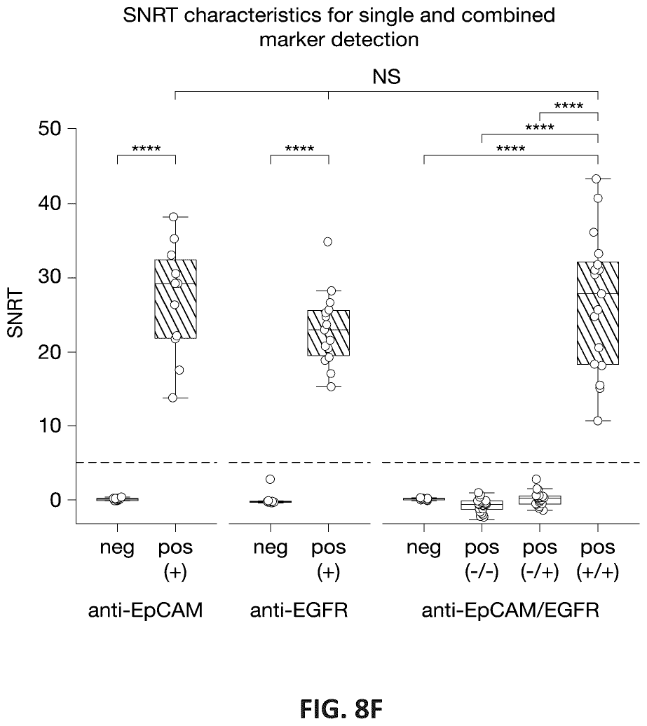

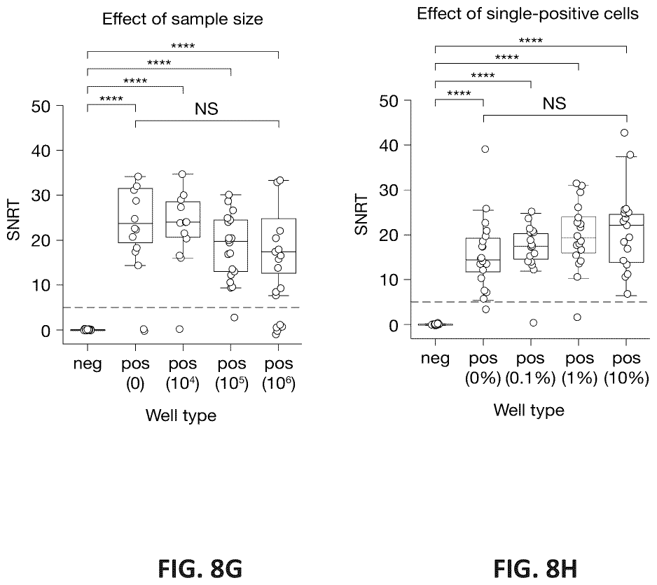

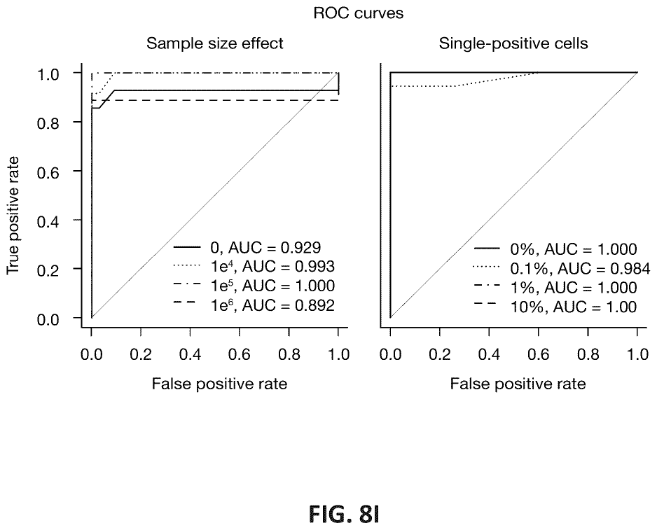

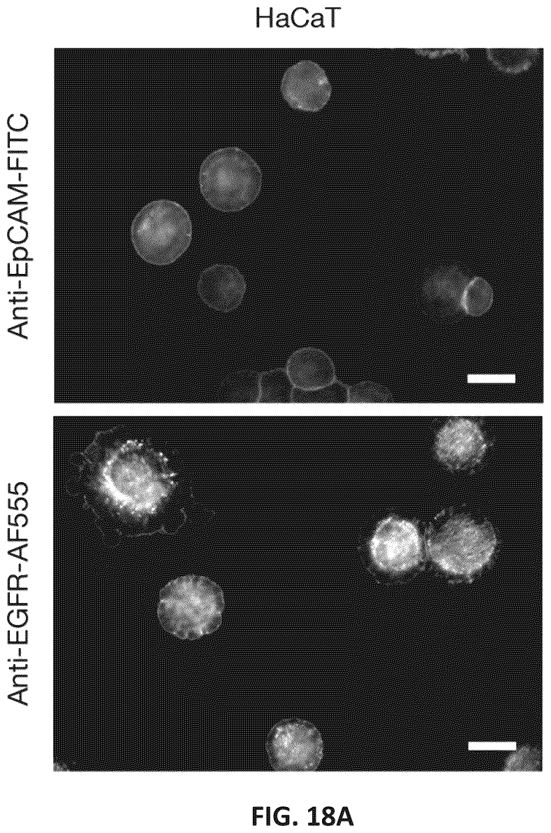

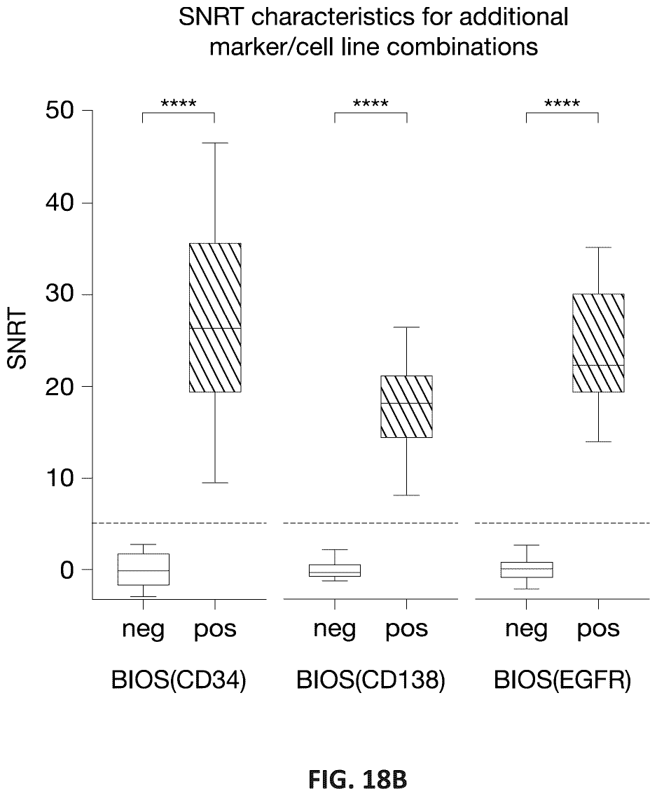

Biocytometry's capabilities in the accurate enumeration of targeted cell immunophenotypes were demonstrated in the HaCaT cell line, an immortalized keratinocyte model characterized by the expression of EpCAM and EGFR membrane proteins (FIG. 18A). Comparable results were obtained for a broad collection of cell lines, such as HeLa, Jurkat, RPMI8226 and KG1a, characterized by unique sets of surface markers (FIG. 18B). Target cells were labeled with ATTO425-maleimide (FIG. 8A) and resuspended in 100 μl of PBS+0.1% gelatin at quantities ranging from 0 to ~1000 cells/sample. Analysis was performed using BIOS(EpCAM), in accordance with the biocytometry workflow (FIG. 6). Luminescence readout was conducted using an optical bottom 96-well plate, followed by microscopic examination to facilitate the absolute quantification of target cells for subsequent data analysis (FIG. 8B). The normalized luminescence values derived from the assay readout were plotted against the number of target cells within each tested sample (FIG. 8C). Biocytometry reproduced the microscopy results with high accuracy (R=0.990, p≈0). To further examine the acquired data, a well-level analysis with a single target cell was conducted. Wells devoid of target cells constantly remained within 3 standard deviations of the blank wells, with the maximum signal-to-noise ratio (SNR) per well reaching, attesting to the inherent specificity of the method. A mean SNR normalized by target cell count (SNRT) was established at 27.1 (FIG. 8D). To evaluate the dynamic range, a series of samples containing varying numbers of HaCaT target cells (EpCAM+, ranging from 0 to 1×10³/sample) was processed using the BIOS(EpCAM) and biocytometry workflow. Statistical analysis revealed consistent assay performance across the range of target cell counts (ANOVA, p=0.930), with a mean SNRT value of 21.1. This value was subsequently utilized as the predictive parameter for target cell number estimations. The signal in wells devoid of target cells remained stable, exhibiting an average SNR≈0 (FIG. 8E).

While the identification of specific cellular types can be achieved using a single surface marker, a more comprehensive identification typically requires multimarker analysis. For multimarker analysis, EpCAM and EGFR were used as the target surface markers. Prior to the combined utilization of EpCAM and EGFR, an experiment was performed to verify the absence of marker bias within the presently described platform (FIG. 8F, left and center). Following the established protocol, target cell quantification was performed with fluorescently labeled HaCaT cells (EpCAM+/EGFR+, ~30 cells/sample), utilizing either BIOS(EpCAM) or BIOS(EGFR). The data demonstrated no statistically significant difference in the performance of the two marker-specific computing units (two-sided t test, p=0.114). Both BIOS(EpCAM) and BIOS(EGFR) supported robust detection of the target cells (Mann-Whitney U test, p<10⁻⁴), with mean SNRT values of 27.0 and 23.0, respectively. Having established equivalent performance for BIOS(EpCAM) and BIOS(EGFR) computing unit systems, both were applied simultaneously in dual-marker detection assays (FIG. 8F, right). For this purpose, target cells (~30 cells/sample) from various cell lines were assessed: HaCaT (EpCAM+/EGFR+), HeLa (EpCAM−/EGFR+), and HL-60 (EpCAM−/EGFR−), employing BIOS(EpCAM+EGFR) in the biocytometry workflow. Utilizing the two markers in combination yielded a detection performance on par (SNRT=26.7; Mann-Whitney U test, p<10⁻⁴) with the use of either marker individually (ANOVA, p=0.298). Importantly, cells devoid of expression for both markers remained under the threshold for positive classification.

### Example 3. Limits of Target Cell Identification in Mixed Samples

In Examples 1-2, single-target sensitivity was established. To evaluate the upper limit of sample size amenable to single-cell resolution, an experiment using the HL-60 cell line was conducted—a suitable proxy for the leukocyte fraction commonly examined in clinical settings. A constant number of HaCaT target cells (EpCAM+, ~30 cells/sample) was introduced while varying the quantity of HL-60 background cells (EpCAM−, 0 to 1×10⁶/sample), and these samples were processed using BIOS(EpCAM) following the biocytometry workflow. The data suggest that assay performance remained stable across varying background cell concentrations (ANOVA, p=0.137; Mann-Whitney U test, p<10⁻⁶), with mean SNRT values of 31.3, 28.3, 22.0, and 24.2 for samples with 0, 1×10⁴, 1×10⁵, and 1×10⁶ background cells, respectively (FIG. 8G). Notably, HL-60 cells on their own did not produce any discernible signal (max SNR=0.3). The assay exhibited high sensitivity, with AUC values of 0.929, 0.993, 1.000, and 0.892 recorded for samples containing 0, 1×10⁴, 1×10⁵, and 1×10⁶ background cells, respectively (FIG. 8I, left). With a sensitivity allowing the identification of one target cell among 1×10⁶ total cells, biocytometry could substantially enhance the routine detection of rare cellular subpopulations, such as hematopoietic stem cells, CAR-T cells, or distinct CD4+ or CD8+ T-cell clonotypes, beyond the conventional limit of 1×10⁻⁴ set by flow cytometry.

To evaluate the influence of single-marker positive subpopulations on the resolution of dual-marker positive target cells, HeLa cells were introduced into the HL-60 background. Samples were prepared with a fixed count of HaCaT target cells (EpCAM+/EGFR+, ~30 cells/sample) while varying the number of HeLa cells (EpCAM−/EGFR+, 0 to 1×10⁻⁴/sample), all against a constant background of HL-60 cells (EpCAM−/EGFR−, 1×10⁵ cells). The samples were then processed as per the standard protocol using BIOS(EpCAM+EGFR). No change in assay performance was observed (ANOVA, p=0.280; Mann-Whitney U test, p<10⁻⁷) despite the increased count of single-positive cells (FIG. 811). Mean SNRT values of 16.7, 17.4, 20.0, and 20.6 were obtained for samples containing 0, 1×10², 1×10³, and 1×10⁴ single-positive cells, respectively. Importantly, the presence of HeLa cells did not yield nonspecific signals, resulting in a maximum SNR of 0.4, comparable to the HL-60 background sample maximum of 0.3. ROC analysis of the assay yielded AUC values of 1.000, 0.984, 1.000, and 1.000 across samples containing 0, 1×10², 1×10³, and 1×10⁴ single-positive cells, respectively (FIG. 8I, right). These results confirmed the effective use of two distinct computing unit types for primary and secondary markers, with assay performance remaining consistent even when partial target cell population constitutes up to 10% of the total sample, a fraction within observed ranges for immune cell subsets.

### Example 4. Detection of Target Cell Subpopulation

This experiment was performed to demonstrate the presently described systems and methods maintain single target sensitivity in the presence of high background.

HL-60 cell line was retrieved from the 37° C. CO₂ incubator. 5 ml of the cell suspension was transferred into a 15 ml tube, spun down at 200 RCF for 5 minutes. The supernatant was removed, and the cells were resuspended in 1 ml of the staining buffer (1×PBS+0.1% Gelatin+0.1% BSA). The cell density was established using the Burker Chamber, and adjusted to 5×10⁶ cells/mL using the staining buffer.

An aliquot of HaCaT cell line was retrieved from liquid nitrogen and thawed out in a 37° C. water bath. The HaCaT aliquot was transferred into a microcentrifuge tube with 1 ml of the staining buffer, and spun down at 200 RCF for 2 minutes. The supernatant was removed, and the cells were resuspended in 1 ml of staining buffer, making the cell density 1×10⁶ cells/mL. A set of aliquots of decreasing concentration of HaCaT cells were prepared in the staining buffer (dilution factor=10).

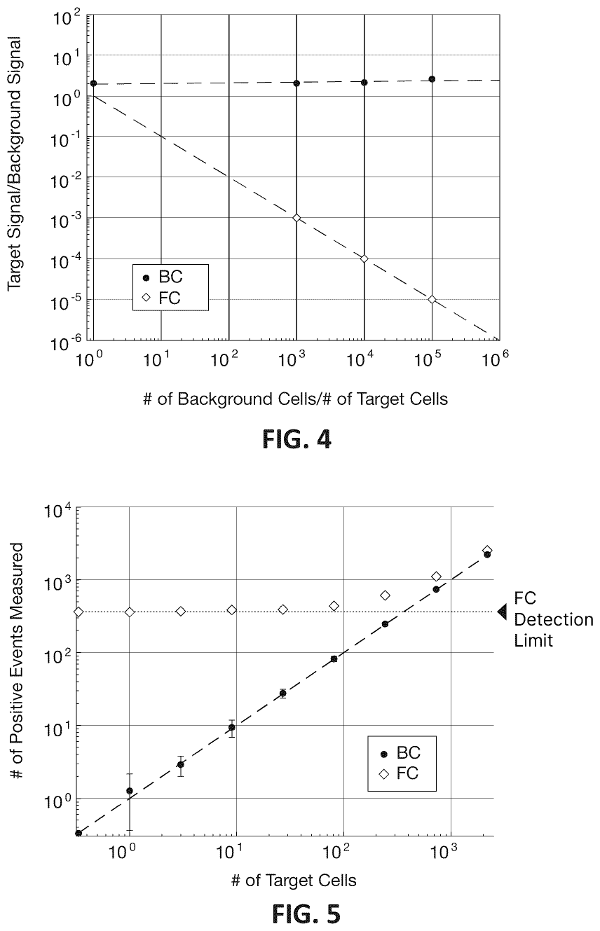

The HL-60 cell suspension and HaCaT cell suspension were combined to obtain a dilution series with target-to-background cell ratios as shown in FIG. 4. Each point in the dilution series had a constant number of HL-60 cells (background cells), which was 100,000. The volume of each sample in the dilution series was adjusted to 100 μL with the staining buffer.

Anti-EpCAM and anti-CD69 primary antibodies were added to all samples in the dilution series. The samples were vortexed and incubated on a rotator for 1 hour at room temperature. After the incubation, the samples were spun down at 200 RCF for 2 minutes, the supernatant was removed, and the cells were resuspended in 500 μL of the staining buffer. This process was repeated twice. After the last supernatant removal, the cells were resuspended in 200 μL of the staining buffer. The samples were divided into two, one for assessment with the presently described methods, and one for assessment with the molecule-based enzymatic method with HRP.

For the assessment with the molecule-based enzymatic method with HRP, 1 μL of HRP-conjugated secondary antibody was added to all samples in the dilution series. The samples were vortexed and incubated on a rotator for 30 minutes at room temperature. The samples were spun down at 200 RCF for 2 minutes, the supernatant was removed, and the samples were resuspended in 500 μL of the washing buffer (1×PBS+0.1% Gelatin+0.1% BSA+0.1% Tween-80). This process was repeated three times. After the last supernatant removal, the cells were resuspended 100 μL of the washing buffer. The contents of each sample were transferred into a 96-well plate. 100 μL of 1-Step™ Turbo TMB ELISA substrate solution was added to each well and mixed. The 96-well plate was incubated for 30 minutes at room temperature. 100 μL of ELISA Stop Solution was added to each well. The absorbance was measured at 450 nm.

For the assessment with the presently described system (FIG. 6), a 96-well plate was placed in a −20° C. freezer, and a thermoblock was set to 40° C. Aliquots of the Reaction Medium was transferred to the 40° C. thermoblock. Aliquots of the frozen Reaction Reagent were thawed in an aluminum block for 3 minutes by transferring them from −80° C. freezer directly to 30° C. incubator. 5 μL of the Reaction Reagent was added to each sample from the above dilution series. The samples were spun down at 200 RCF for 1 minute for 10 times, rotating tubes 180 degrees after each minute. The samples were resuspended and placed on a shaker at 1750 rpm, acceleration 2, for 30 seconds. 70 μL of each sample was added to the respective Reaction Medium tubes. The tubes were inverted to mix. For each sample, 100 μL was dispensed per well in one column by reverse pipetting beginning at A2 in the 96-well plate. The 96-well plate was incubated at 4° C. for 10 minutes, and then 30° C. for 4 hours. The readout buffer was prepared by mixing 11.4 mL of Luciferase buffer, 300 μL of 0.1% cycloheximide stock, and 3000 μL of furimazine. 100 μL of the readout buffer was added to each well, and the luminescence was measured.

As shown in FIG. 4, the presently described biocytometry system (BC) exhibited an all-or-nothing behavior, rejecting low expressing background cells and recognizing all high expressing target cells equally. By doing so, the presently described system maintained single target sensitivity in the presence of high background. The molecule-based enzymatic method (Fc) with HRP measured total antigen and therefore the target signal strength was diminished as the background increased. Therefore, the presently described systems and methods maintained single target sensitivity in the presence of high background.

### Example 5. Quantification of Spiked Target Cell Subpopulation

This experiment was performed to demonstrate the presently described systems and methods exhibit exceptional false positive rate characteristics.

The cultivation medium was prepared in a 15 mL tube with RPMI, 10% FBS, 1× antibiotic/mycotic solution. The tube was transferred into a 37° C. water bath.

An aliquot of PBMCs were retrieved from liquid nitrogen, and incubated in a 37° C. water bath for 10 minutes. 1 mL of the cultivation medium was slowly added to the PBMC aliquot, and the PBMCs were mixed by slow pipetting. The PBMC suspension was transferred into a 15 mL tube, spun down at 350 RCF for 5 minutes, and the supernatant was removed. The PBMC pellet was resuspended in the 200 μL staining buffer (1×PBS+0.1% Gelatin+0.1% BSA). The PBMC suspension was filtered through a 20 m filter. The PBMC cellular density was established using a Burker chamber. The original PBMC suspension was diluted 1:20, and stored at 4° C.

An aliquot of HaCaT cells were retrieved from liquid nitrogen, and incubated in a 37° C. water bath for 5 minutes. The HaCaT cell aliquot was transferred into a microcentrifuge tube containing 1 mL of the staining buffer and spun down at 200 RCF for 2 minutes. The supernatant was removed, and the cells were resuspended in 1 mL of the staining buffer (1×10⁶ cells/mL). A set of aliquots with decreasing concentration of HaCaT cells in staining buffer were prepared (dilution factor=10).

The PBMC suspension and HaCaT cell suspension were combined to obtain a dilution series with serial target-to-background cell ratios. Each point in the dilution series had a constant number of PBMC (background cells), which was 500,000. The volume of each sample in the dilution series was adjusted to 200 μL with the staining buffer. The samples were divided into two, one for assessment with the presently described methods, and one for assessment with the instrument-based brute force method (flow cytometry).

For the assessment with the flow cytometry, the samples were transferred into a standard 5 mL PS tube. 1 μL of anti-EpCAM primary antibody was added to the tube. The tube was vortexed and incubated on a rotator for 30 minutes at room temperature. The whole sample was acquired with a flow cytometer.

For the assessment with the presently described system (FIG. 6), a 96-well plate was placed in a −20° C. freezer, and a thermoblock was set to 40° C. Aliquots of the Reaction Medium was transferred to the 40° C. thermoblock. Aliquots of the frozen Reaction Reagent were thawed in an aluminum block for 3 minutes by transferring them from −80° C. freezer directly to 30° C. incubator. 5 μL of the Reaction Reagent was added to each sample from the above dilution series. The samples were spun down at 200 RCF for 1 minute for 10 times, rotating tubes 180 degrees after each minute. The samples were resuspended and placed on a shaker at 1750 rpm, acceleration 2, for 30 seconds. 70 μL of each sample was added to the respective Reaction Medium tubes. The tubes were inverted to mix. For each sample, 100 μL was dispensed per well in one column by reverse pipetting beginning at A2 in the 96-well plate. The 96-well plate was incubated at 4° C. for 10 minutes, and then 30° C. for 4 hours. The readout buffer was prepared by mixing 11.4 mL of Luciferase buffer, 300 μL of 0.1% cycloheximide stock, and 3000 μL of furimazine. 100 μL of the readout buffer was added to each well, and the luminescence was measured.

As shown in FIG. 5, target cell dilution series was measured by the presently disclosed biocytometry system (BC) and flow cytometry (FC). Each of sex independent samples included varying amounts of target cells (HaCaT, EpCAM⁺), spiked into PBMC sample matrix. Excellent correlation with the dilution series was attained with both the presently disclosed system (R=0.9961) and flow cytometry (R=0.9931) in low sensitivity measurements, with the presently disclosed system having exceptional false positive rate characteristics (0 vs. 899 false positive events per million cells analyzed for the presently disclosed system and flow cytometry, respectively). Therefore, the presently described systems and methods exhibited exceptional false positive rate characteristics.

### Example 6. Comparative Assessment Across Complex Sample Matrices

Biocytometry and flow cytometry were further compared herein on a large set of clinically relevant sample matrices.

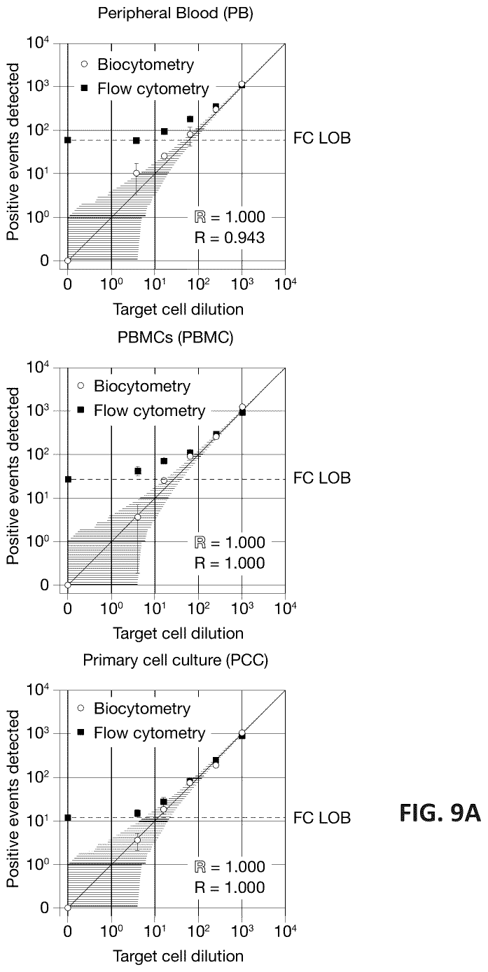

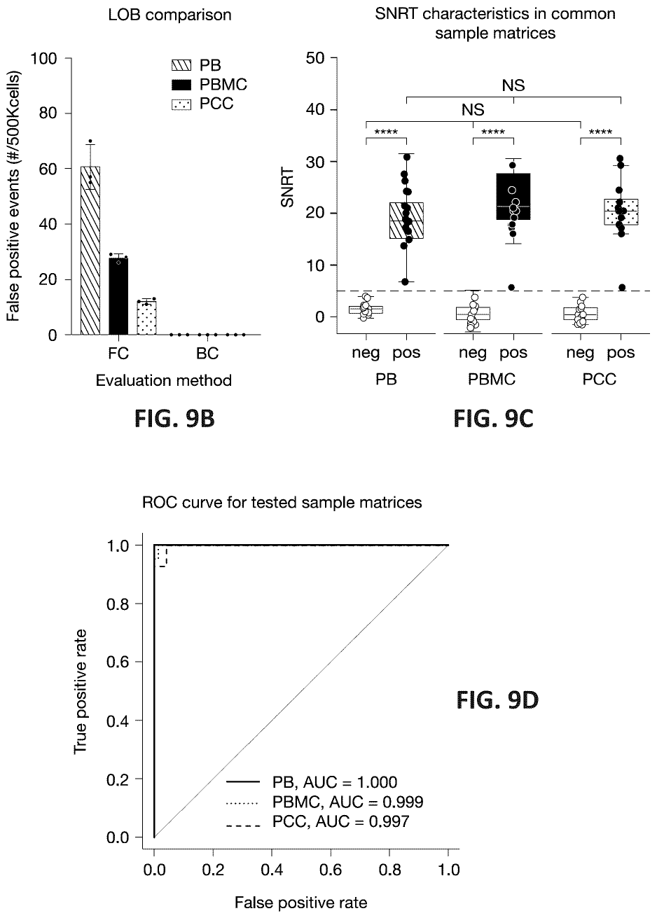

A set of samples was prepared by introducing varying quantities of unlabeled HaCaT cells (EpCAM+, 0, 4, 16, 64, 256, 1024/sample) into a variety of sample matrices, which included peripheral blood (FIG. 9A, top), cryopreserved PBMCs (FIG. 9A, center), and primary cell cultures (FIG. 9A, bottom). Each sample contained the equivalent of 1×10⁶ leukocytes. The resulting samples were subsequently split and processed separately. Biocytometric analysis utilized BIOS(EpCAM), adhering to the biocytometry workflow. Concurrently, flow cytometry samples were stained with AF647-conjugated anti-EpCAM antibody, following the flow cytometry workflow. Upon analysis, a strong correlation was observed between the two methods in peripheral blood (R=1.000, p≈0 for biocytometry; 0.943, p≈0 for flow cytometry), cryopreserved PBMCs (R=1.000, p≈0 for biocytometry; R=1.000, p≈0 for flow cytometry) and primary cell cultures (R=1.000, p≈0 for biocytometry; R=1.000, p≈0 for flow cytometry). For samples with lower target cell counts (0-64 spiked target cells, <10⁻⁴ sensitivity), flow cytometry (FC) analysis was impeded by its limit of blank (LOB), failing to quantify below this level. Significant variability in flow cytometry LOB was observed across examined sample matrices, with cryopreserved PBMCs yielding the highest false-positive rate at 121 per million cells analyzed, followed by lysed blood at 55 per million and primary cell cultures at 31 per million. Biocytometry (BC), on the other hand, maintained a null LOB across all tested sample matrices (FIG. 9B).

To validate the presently described biocytometry platform's capacity for absolute quantification in complex sample matrices, a constant number of fluorescently labeled HaCaT target cells (EpCAM+, ~30 cells/sample) was introduced into a variety of sample matrices, including peripheral blood, cryopreserved PBMCs, and primary cell cultures. Each sample contained the equivalent of 5×10⁵ leukocytes. The analysis was performed with BIOS(EpCAM), adhering to the biocytometry workflow. Microscopic examination was performed to confirm the exact count of HaCaT cells in a sample. The assay's performance was uniform (ANOVA, p=0.991; Mann-Whitney U test, p<10⁻⁴) in all evaluated matrices, as signified by mean SNRT values of 17.9, 21.6, and 19.3 for peripheral blood (PB), cryopreserved PBMCs, and primary cell cultures (PCC), respectively (FIG. 9C). Consistency across different matrices was further validated by the ROC curves, which displayed AUC values of 1.000 for peripheral blood, 0.999 for cryopreserved PBMCs, and 0.997 for primary cell cultures (FIG. 9D).

### Example 7. Quantification of Leukocyte Activation in PBMCs

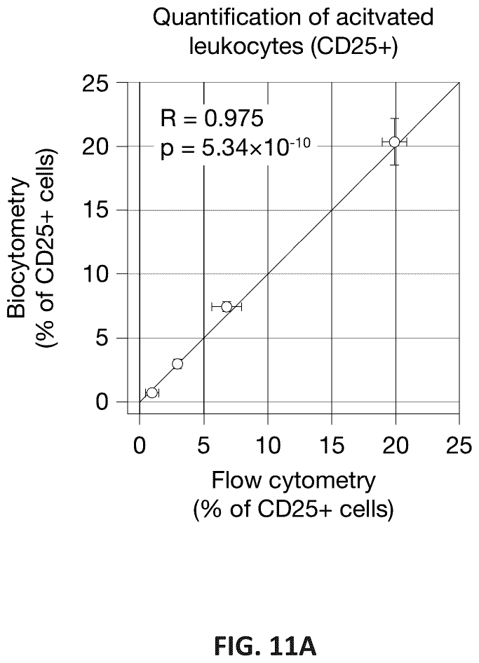

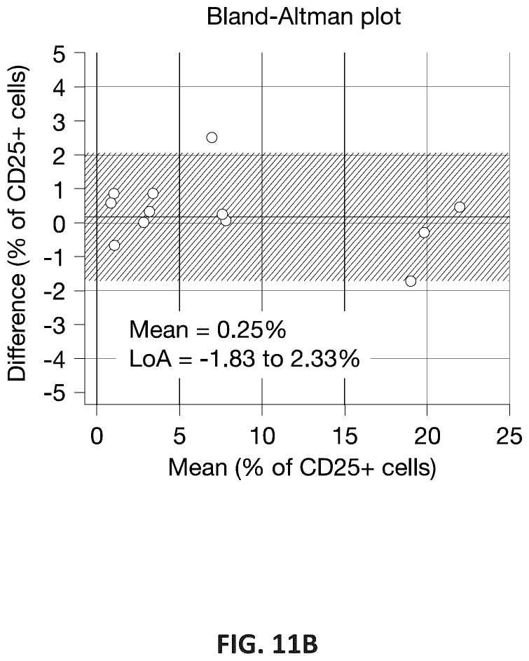

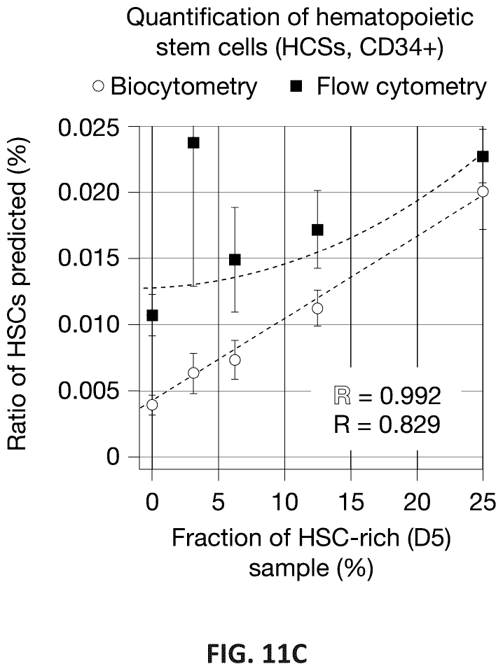

The presently described biocytometry and flow cytometry were further compared herein on a large set of clinically relevant sample matrices. Biocytometry was evaluated for the quantification of leukocyte activation status. CD25 is a putative marker for long-term leukocyte activation. The extent of leukocyte activation in assays can range from low levels when evaluating particular T-cell receptor (TCR) subsets to nearly universal when assessing systemic immune function. To obtain a high activation rate, a PBMC sample from a healthy donor was treated with the nonspecific mitogen PHA (2.5 μg/ml) alongside an uninduced control sample. To capture varying activation rates, the induced and uninduced control samples were combined at different ratios (0%, 11%, 33% and 100% induced). Each sample contained the equivalent of 1×10⁶ leukocytes. The resulting dilution series was evaluated in parallel by biocytometry and flow cytometry. In compliance with the biocytometry workflow, the biocytometry assay utilized BIOS(CD25), while flow cytometry was performed according to the flow cytometry protocol using FITC-conjugated anti-CD25 antibodies. A robust correlation was observed between biocytometry and flow cytometry for estimating CD25+ events, as indicated by a Pearson correlation coefficient of R=0.975 (p<10⁻⁹) (FIG. 11A). To assess the degree of agreement between the two methodologies, Bland-Altman analysis was performed. The resultant plot disclosed a mean bias of 0.25%, with limits of agreement spanning from −1.83% to 2.33% (FIG. 11B).

### Example 8. HSC Enumeration in Mobilized Patients

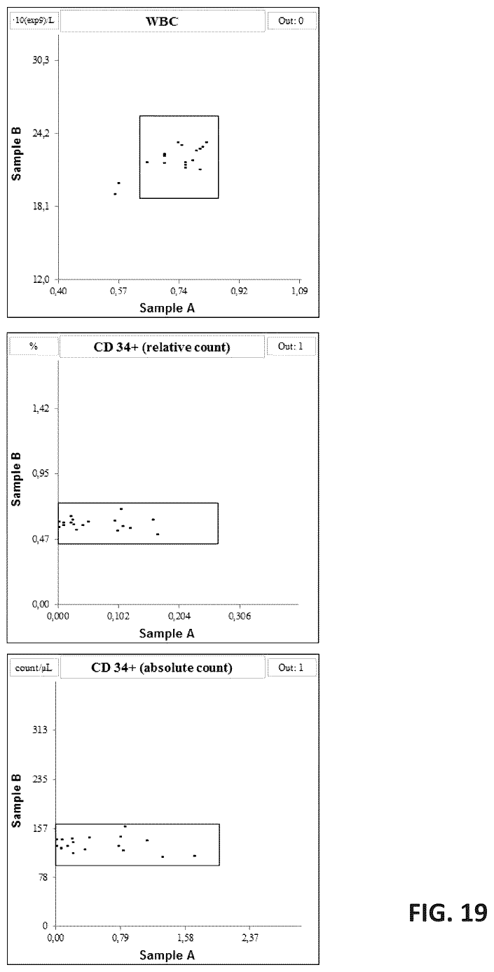

The presently described biocytometry was evaluated for the enumeration of CD34+ hematopoietic stem cells (HSCs), a critical indicator in stem cell mobilization procedures. Patients receiving granulocyte colony-stimulating factor (G-CSF) for stem cell mobilization provided peripheral blood samples both at the onset of mobilization (DO) and at the time of leukapheresis (D5). PBMCs were isolated via conventional Ficoll density gradient separation. A dilution series was established using DO and D5 samples, generating a range of samples with increasing HSC content. Each sample contained the equivalent of 1×10⁶ leukocytes. The samples were subjected to parallel analysis using a biocytometry workflow with BIOS(CD34) and flow cytometry following a quantification protocol using PE-conjugated anti-CD34 antibodies. The biocytometry results exhibited a strong linear relationship with the HSC dilution series (R=0.992, p≈0) (FIG. 11C). Conversely, flow cytometry yielded poor correlation (R=0.829, p≈0), a limitation attributable to HSC concentrations nearing the detection limit (FIG. 11C). The two methods exhibited comparable coefficients of variation (CVs): 18.4% for biocytometry and 24.9% for flow cytometry. Flow cytometry showed a notably higher maximum CV (45.2%) than biocytometry (25.0%) at the lowest HSC concentration. The observed limit of detection (LOD) aligns with the baseline HSC levels in healthy populations and was consistent with values reported in ISHAGE interlaboratory harmonization studies (FIG. 19).

### Example 9. Detection of Apoptotic EGFR Positive Cells

This experiment was performed to detect EGFR positive cells with the presently described systems and methods.

In a 12-well plate, 5×10⁴ HaCaT cells were seeded in DMEM++ medium (DMEM medium, Fetal Bovine Serum, and stabilized antibiotic antimycotic solution). The cells were grown for 2 days at 37° C. in a cell culture incubator. Before inducing the cell culture, the medium was changed to remove all cells in suspension. Apoptosis was induced using multiple concentrations of Ara-C (cytosine beta-D-arabinofuranoside) (1000 μM, 200 μM, 40 μM, 8 μM). ddH₂O was used as a control. The plate was gently swirled to mix, and the cells were incubated for 16 hours at 37° C. in a cell culture incubator.

The supernatant was removed, and the plate was washed with DPBS by gently swirling the plate. 1 mL of 1× stable cell (5× Stable Cell Trypsin solution (Sigma) diluted with DPBS) was added and incubated for 5 minutes at 37° C. The stable cell solution was removed. The plate was incubated for 10 minutes at 37° C. without medium DPBD, and the plate was vortexed for 1 minutes at 1200 rpm and incubated for 5 minutes at 37° C. The plate was vortexed for 2 minutes to detach the cells. DMEM++ was added to stop the trypsinization process. All supernatant was transferred to a tube. The samples were treated with DNase I and shook for 5 minutes at 1400 rpm at 37° C. using a thermoblock. The cells were filtered using 20 m filter. The cells were stained with 2 μM of atto-425-mal, incubated for 10 minutes on a rotator in the dark, and spun down at 250 g for 1 minutes, rotating tubes 180 degrees. The spin-down was repeated, and the supernatant was removed. The cells were resuspended in Annexin Buffer (25 nM HEPES, 140 NaCl, 2.5 mM CaCl₂, 0.1% P188, and 1% yeast extract, buffered to pH 7.4) to concentrate the samples. The cells were counted using a Burker chamber and the volume was adjusted to 2,000 cells per 1 mL. The samples were stored at 4° C. until later use.

Seven Master Tubes (alginic acid sodium salt, gelatin from porcine skin, 1×YPD (yeast extract, peptone, glucose, and ddH₂O)) (1 for each sample and blank) was preheated at 40° C. for at least 10 minutes. The seven Apoptosis Reaction Tubes (comprising Reaction Reagent, e.g., strain Annexin, strain EGFR, strain BAR1, DPBS, trehalose) were spun down at 200 g for 30 seconds.

Strain Annexin
(SEQ ID NO: 1)
BY4741 MATa Δbar1 Δmfa1 Δmfa2 Δmfx1 Δmfx2 Δaga2 GPA1(468-472)::DSGIL
pFIG1-LUC 14xpFIG1-ScMFA pTDH3-OMH-Annexin-FLAG-FLO11
Strain EGFR
(SEQ ID NO: 2)
BY4742 MATx Δmfx1 Δmfx2 pRAD27-SST2 GPA1(468-472)::DSGIL pFIG1-LUC
8xpFIG1-MFX pTDH3-OMH-EGFR-HRL-FLAG-FLO11
Strain BAR1
(SEQ ID NO: 3)
BY4741 MATa Δbar1 Δmfa1 Δmfa2 Δaga2 pTDH3-OMH-3X(FLAG-HRL)-HRL-
FLO11 pTDH3-BAR1-FLO11

Each sample was added to an Apoptosis Reaction Tube comprising Reaction Reagent to rehydrate the contents. Annexin Buffer was added to last Apoptosis Reaction Tube for blank. Apoptosis Reaction Tubes were incubated at room temperature for 1 minute. The reaction was resuspended on a shaker at 1400 rpm for 1 minute. The tubes were spun down at 200 g for 1 minute, twisting the tubes 180 degrees from its initial position within the centrifuge, and this process was repeated for five times. The reaction was resuspended on a shaker at 1400 rpm for 1 minute.

For each sample and blank, 70 μL of each reaction from the Apoptosis Reaction Tubes were transferred into each Master Tubes. The content was mixed gently by inverting the Master Tubes and chilled at −20° C. for 10 minutes. From each Master Tube, 100 μL were dispensed into 1 column of wells of the pre-chilled 96-well plate. The plate was incubated at 4° C. for 10 minutes. The plate was transferred to 30° C. incubator and incubated for 4 hours.

The plate was heated at 40° C. for 10 minutes. 100 μL of Luciferase Buffer (1M Tris adjusted to pH 8.5 and ddH₂O) was added to each well. The plate was vortexed at 1600 g for 3 minutes. Luminescence was measured on a plate reader. For each sample well, percent apoptotic cells and EGFR+ cells were computed using the following formula:

$$\% targets = \frac{{Luminescence}_{sample} - {Luminescence}_{blank}}{3 \times 200 \times {Luminescence}_{blank}}$$

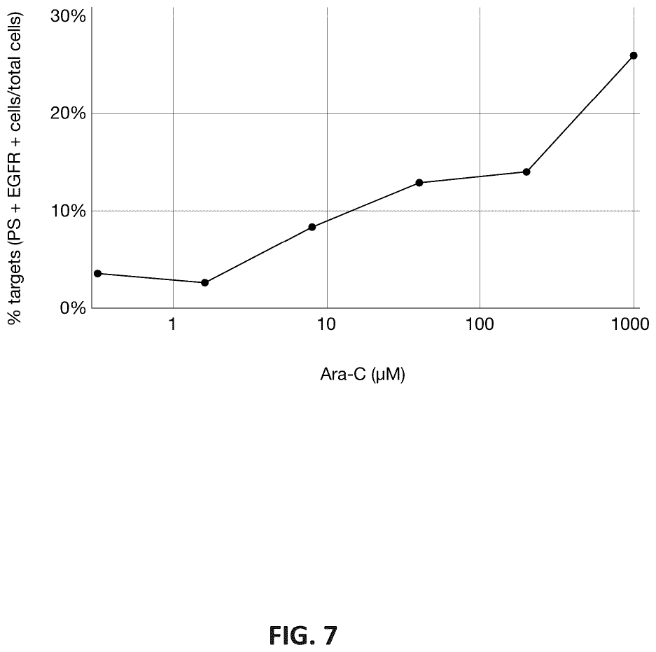

Enumeration of EGFR positive cells apoptotic cells in Ara-C induced samples are shown in FIG. 7.

### Example 10. Platform Compatibility with Challenging Sample Matrices

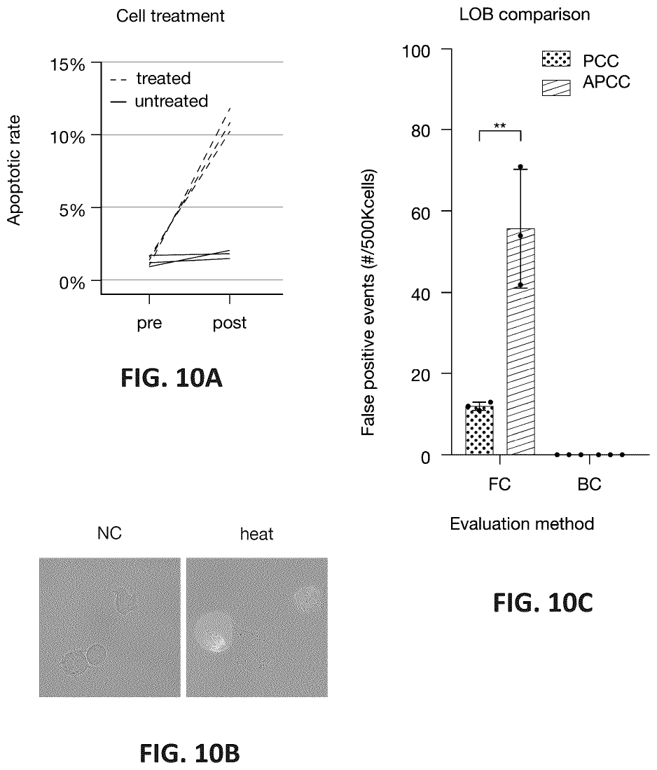

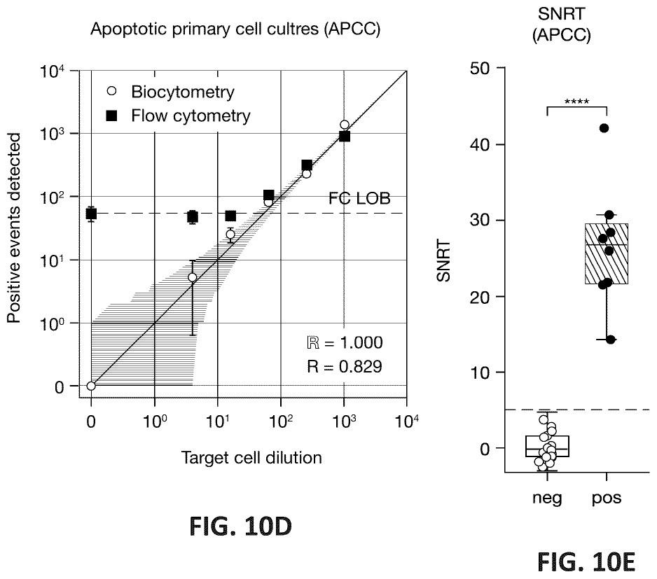

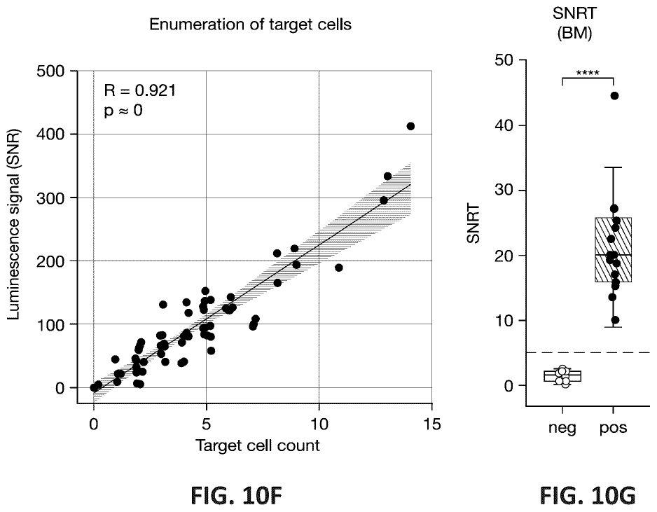

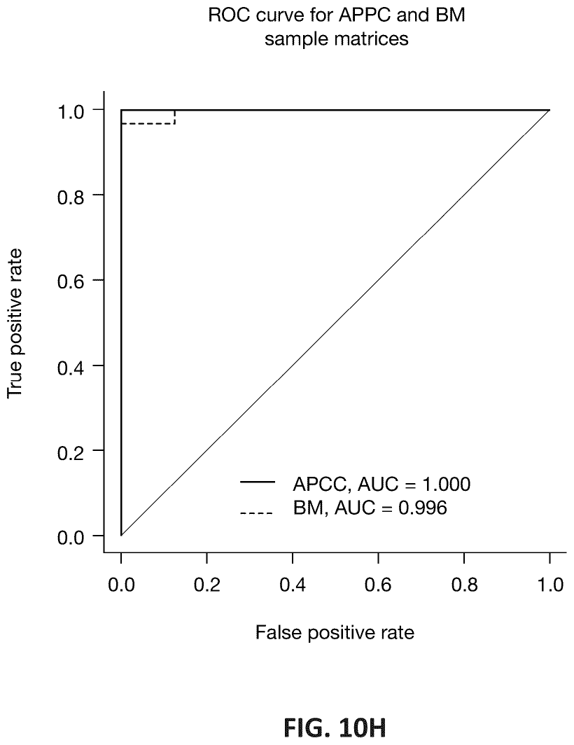

The presently described biocytometry system was evaluated in the analysis of primary cell cultures exhibiting a high fraction of apoptotic cells. Apoptotic cells, arising from various factors such as sample age, treatment, or manipulation, present analytical challenges in the analysis. Apoptosis in primary cell cultures was induced via brief heat shock. The apoptotic rate of the cell cultures rose significantly, with 12.3% of cells identified as apoptotic (FIG. 10A), as confirmed by fluorescence staining using Annexin V FITC conjugate (FIG. 10B). A set of samples was prepared by introducing varying quantities of unlabeled HaCaT cells (EpCAM+, 0, 4, 16, 64, 256, 1024/sample) into a suspension of apoptotic primary cells. Each sample contained the equivalent of 1×10⁶ leukocytes. The resulting samples were subsequently split and processed separately. Biocytometric (BC) analysis was performed with BIOS(EpCAM), adhering to the biocytometry workflow. Concurrently, flow cytometry (FC) samples were stained with an AF647-conjugated anti-EpCAM antibody, following the flow cytometry workflow. Flow cytometry analysis revealed a marked 464% escalation in false-positive events, with a corresponding LOB increase to 144 false-positive events per million cells analyzed (FIG. 10C). In stark contrast, biocytometry maintained a null LOB, consistent with prior findings. Linear regression demonstrated a strong correlation for biocytometry (R=1.000, p≈0), in contrast to the suboptimal performance of flow cytometry (R=0.829, p=0.042) (FIG. 10D). During the identification of fluorescently labeled HaCaT cells, biocytometry exhibited nominal performance (Mann-Whitney U test, p<10⁻⁴), registering a mean SNRT of 25.6 and an AUC of 1.000 in ROC analysis (FIGS. 10E and 10H). Biocytometry was evaluated for the analysis of bone marrow aspirates. Bone marrow is especially difficult to analyze due to the complex microenvironment, debris, and small sample size. To explore whether biocytometry could mitigate these challenges, bone marrow samples from a patient undergoing hematopoietic stem cell transplantation (HSCT) for acute myeloid leukemia (AML) were spiked with fluorescently labeled HaCaT target cells (0-30 cells/sample). The samples were analyzed using BIOS(EpCAM). Given the limited sample, flow cytometry analysis was not conducted. The experimental data showed an unaltered baseline LOB, along with a significant correlation (R=0.872, p≈0) between target cell counts estimated by the biocytometry platform and those confirmed through fluorescence microscopy (FIG. 10F). Furthermore, the target cell detection performance was proved to be comparable to that in other sample matrices (Mann-Whitney U test, p<10⁻⁴), as indicated by a mean SNRT of 20.0 (FIG. 10G). The ROC curve for the assay yielded an AUC value of 0.996 (FIG. 10H).

### Example 11. Measurement of the Cell-Specific Apoptosis Rate

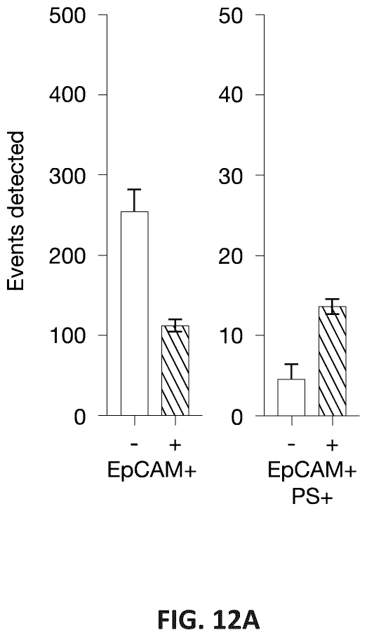

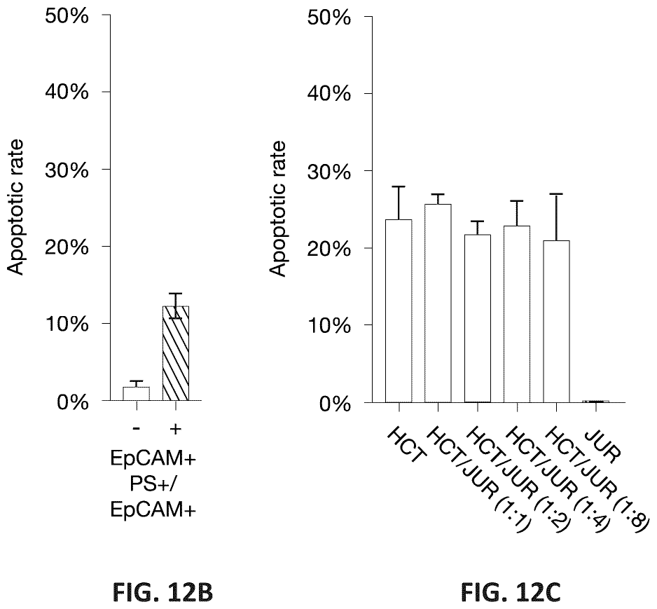

The presently described biocytometry was evaluated for measuring cell-specific apoptosis in mixed cell cultures. Despite the growing trend toward studying apoptosis in complex biological systems such as organoids and mixed cell cultures, conventional methods such as Caspase-based assays and MTT tests still yield limited sensitivity and quantify only aggregate apoptosis levels. To assess cell-specific apoptosis, mixed cultures of HaCaT (HCT, EpCAM+) and Jurkat (JUR, EpCAM−) cells at varied ratios (1:0 to 1:8) were utilized. Cells were cultured at a density of 40,000 cells/well in RPMI medium supplemented with 10% FBS and either subjected to 1 mM DTT treatment or left untreated. Following a 2.5-hour incubation at 37° C. in a 5% CO₂ atmosphere, both total and apoptotic HaCaT cell counts were established using BIOS(EpCAM) and BIOS (EpCAM+PS), adhering to the biocytometry protocol. A noticeable reduction in total EpCAM+ cell count was evident in induced cell cultures, alongside an increase in the number of apoptotic EpCAM+ cells, aligning with the anticipated apoptotic changes (FIG. 12A). Apoptotic rates were calculated by normalizing BIOS(EpCAM+PS) counts to total cell counts from BIOS(EpCAM). A 7.2-fold increase in apoptotic rates among EpCAM+ cells exposed to 1 mM DTT was observed, reaching 12.2% compared to a 1.7% baseline in untreated controls (FIG. 12A). Importantly, the apoptotic rate remained consistent across varying Jurkat cell ratios, underscoring the assay's specificity for HaCaT cells, which was further corroborated by the absence of measurable apoptotic events in HaCaT-devoid samples (FIG. 12C).

Exemplary polynucleotides and amino acid sequences of one or more genes described in the present disclosure are shown in the accompanying Sequence Listing.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A method of detecting presence or absence of an output signal indicative of a characteristic of an input sample, comprising:
(a) contacting at least one sample object derived from the input sample with a Reaction Reagent comprising at least one computing unit (CU) and with a Reaction Medium, wherein the at least one CU is configured to interact with the at least one sample object;
(b) incubating (a) for a sufficient amount of time in an incubator for the output signal to be generated;
(c) adding a readout reagent comprising a reporter entity to (b), wherein the reporter entity generates the output signal; and
(d) measuring the output signal, wherein the output signal is directly proportional to the amount of the at least one sample object derived from the input sample, thereby detecting the presence or absence of the output signal indicative of the characteristic of the input sample.

2. The method of claim 1, wherein the contacting in step (a) comprises first (i) adding the at least one sample objects to the Reaction Reagent and then (ii) adding the Reaction Medium to (i).

3. The method of any one of claims 1-2, wherein the sufficient amount of time in step (b) is about 4 hours.

4. The method of any one of claims 1-3, wherein the characteristic is apoptosis, activation, suppression, anergy, or differentiation.

5. The method of any one of claims 1-4, wherein the output signal does not comprise any background signal regardless of the input sample type.

6. The method of any one of claims 1-4, wherein the input sample is whole blood, a stabilized leukocyte fraction, isolated PBMCs, cryogenically stored PBMCs, primary cell cultures, cell lines, tumor samples, stem cells, or CAR-T cells.

7. The method of any one of claims 1-6, wherein the Reaction Reagent comprises the at least one CU and hydrogel.

8. The method of claim 7, wherein the hydrogel is gelatin.

9. The method of any one of claims 7-8, wherein the Reaction Reagent further comprises yeast extract, peptone, D-glucose, Dulbecco's phosphate-buffered saline, D-(+)-Trehalose dihydrate, and skim milk.

10. The method of any one of claims 7-9, wherein the Reaction Reagent can be stored up to about 2 weeks at room temperature without loss of signal.

11. The method of claim 10, wherein the Reaction Reagent can be stored up to about 8 weeks at 4° C. without loss of signal.

12. The method of any one of claims 1-6, wherein the Reaction Medium is a semi-permeable nutritive medium with thermoresponsive properties.

13. The method of claim 12, wherein the Reaction Medium comprises hydrogel.

14. The method of claim 13, wherein the hydrogel is gelatin or sodium alginate.

15. The method of any one of claims 12-14, wherein the Reaction Medium further comprises yeast extract, peptone, D-glucose, and ampicillin.

16. The method of any one of claims 1-15, wherein the output signal is based on spectrophotometric property or fluorometric property of the reporter entity.

17. The method of any one of claims 1-16, wherein the reporter entity is a fluorescent protein.

18. The method of any one of claims 1-16, wherein the reporter entity is a luminescent protein.

19. The method of claim 18, wherein the luminescent protein is luciferase.

20. The method of any one of claims 18-19, wherein the readout reagent comprises luminescence substrate and luminescence buffer.

21. The method of any one of claims 18-20, wherein the output signal is luminescence.

22. The method of any one of claims 1-21, wherein the method comprises at least one logical operator module.

23. The method of claim 22, wherein a logical operator module of the at least one logical operator module comprises the at least one sample object and two or more CUs of the at least one CU.

24. The method of claim 22 or 23, wherein the at least one logical operator module generates one or more output signals.

25. The method of any one of claims 22-24, wherein the at least one logical operator module comprises a YES gate, an AND gate, a NAND gate, an OR gate, a NOR gate, a XOR gate, a XNOR gate, a NOT gate, or any combination thereof, wherein the two or more CUs comprise a first CU and a second CU.

26. The method of claim 25, wherein the YES gate comprises generating the one or more output signals only when both the first CU and the second CU are interacting with the at least one sample object.

27. The method of claim 25, wherein the AND gate comprises generating the one or more output signals only when both the first CU and the second CU are interacting with the at least one sample object.

28. The method of claim 25, wherein the NAND gate comprises suppressing or diminishing the one or more output signals when both the first CU and the second CU are interacting with the at least one sample object.

29. The method of claim 25, wherein the OR gate comprises generating the one or more output signals when either the first CU or the second CU or when both the first CU and the second CU are interacting with the at least one sample object.

30. The method of claim 25, wherein the NOR gate comprises generating the one or more output signals when both the first CU and the second CU are not interacting with the at least one sample object.

31. The method of claim 25, wherein the XOR gate comprises generating the one or more output signals when either the first CU or the second CU but not both CUs are interacting with the at least one sample object.

32. The method of claim 25, wherein the XNOR gate comprises generating the one or more output signals when either both the first CU and the second CU or when both the first CU and the second CU are interacting with the at least one sample object.

33. The method of claim 25, wherein the NOT gate comprises suppressing or diminishing the one or more output signals when the first CU is interacting with the at least one sample object.

34. The method of any one of claims 1-33, wherein the sample object independently is a cell or a molecule.

35. The method of any one of claims 1-34, wherein a CU of the at least one CU is independently
(i) associated with one or more surface-bound entities (SBEs);
(ii) capable of recognizing a signal object (SO);
(iii) capable of producing an SO;
(iv) capable of degrading an SO;
(v) capable of producing a change in a material property of the reaction medium;
(vi) capable of producing another CU; or
(vii) capable of changing its state following signal recognition or external influence.

36. The method of claim 35, wherein the CU comprises a molecule or a cell.

37. The method of claim 36, wherein the molecule comprises a monomeric or multimeric molecule.

38. The method of claim 36 or 37, wherein the molecule comprises a polypeptide, a polypeptide derivative, a nucleic acid, or a nucleic acid derivative.

39. The method of claim 38, wherein the polypeptide is an antibody or an enzyme.

40. The method of claim 36, wherein the cell comprises a wildtype cell, a synthetic cell, or an engineered cell.

41. The method of claim 36 or 40, wherein the cell is a yeast cell.

42. The method of claim 41, wherein the yeast cell is an engineered yeast cell.

43. The method of any one of claims 40-42, wherein the CU state change detects cell specific apoptosis.

44. The method of claim 43, wherein an SBE of the one or more SBEs is a phosphatidylserine binding SBE.

45. The method of claim 43, wherein the one or more SBEs are cell-specific, antigen binding SBEs.

46. The method of any one of claims 43-45, the CU of the at least one CU is associated with the phosphatidylserine binding SBE, and one or more CUs of the at least one CU is associated with the cell-specific, antigen binding SBEs.

47. The method of claim 43, wherein the yeast cell comprises Annexin strain, EGFR strain, and BAR1 strain.

48. The method of claim 47, wherein the Annexin strain comprises a polypeptide sequence having at least about 80% sequence identity to SEQ ID NO: 1.

49. The method of claim 48, wherein the Annexin strain comprises a polypeptide sequence of SEQ ID NO: 1.

50. The method of claim 47, wherein the EGFR strain comprises a polypeptide sequence having at least about 80% sequence identity to SEQ ID NO: 2.

51. The method of claim 48, wherein the EGFR strain comprises a polypeptide sequence of SEQ ID NO: 2.

52. The method of claim 47, wherein the BAR1 strain comprises a polypeptide sequence having at least about 80% sequence identity to SEQ ID NO: 3.

53. The method of claim 48, wherein the BAR1 strain comprises a polypeptide sequence of SEQ ID NO: 3.

54. The method of any one of claims 35-53, wherein the CU comprises at least one SBE of the one or more SBEs.

55. The method of claim 35, wherein an SBE of the one or more SBEs comprises a cell surface receptor, a transmembrane protein or a glycophosphatidylinositol (GPI)-anchored protein.

56. The method of claim 35, wherein the SO comprises a bio-generated entity.

57. The method of claim 56, wherein the bio-generated entity comprises a signaling molecule, a metabolite, a peptide, or a synthetic compound.

58. The method of claim 57, wherein the signaling molecule comprises a hormone, a cytokine, an interleukin, a chemokine, or a pheromone.

59. The method of any one of claims 35-58, wherein the SO is produced by a CU of the at least one CU.

60. The method of any one of claims 35-58, wherein the SO is not produced by a CU of the at least one CU.

61. The method of any one of claims 35-60, wherein the CU forms one or more computational clusters.

62. The method of claim 61, wherein a force is applied to the CU to form the one or more computational clusters.

63. The method of claim 62, wherein the force is a centrifugal force, a magnetic force, or an electrostatic force.

64. The method of claim 63, wherein the centrifugal force is a continuous centrifugal force.

65. The method of claim 64, wherein a reaction tube comprising the at least one sample object, the Reaction Reagent, and the Reaction medium is twisted around its z-axis to create the continuous centrifugal force.

66. A system for biological computing, comprising:
(a) at least one sample object derived from an input sample;
(b) a Reaction Reagent comprising at least one computing unit (CU) configured to interact with the at least one sample object;
(c) a Reaction Medium; and
(d) a readout reagent comprising a reporter entity and a reporter buffer.

67. The system of claim 66, further comprising one or more evaluation strips and a plate adopter.

68. The system of claim 66 or 67, further comprising one or more 96-well plates and one or more microcentrifuge tubes.

69. The system of any one of claims 66-68, further comprising a thermocycler.

70. The system of any one of claims 66-69, further comprising a plate reader.

71. The system of any one of claims 66-70, wherein one or more computational clusters are formed when the at least one CU interacts with the at least one sample object.

72. The system of claim 71, wherein a force is applied to form the one or more computational clusters.

73. The system of claim 72, wherein the force is a centrifugal force, a magnetic force, or an electrostatic force.

74. The system of claim 73, wherein the centrifugal force is a continuous centrifugal force.

75. The system of claim 74, wherein a reaction tube comprising the at least one sample object, the Reaction Reagent, and the Reaction medium is twisted around its z-axis to create the continuous centrifugal force.

76. A kit for biological computing, comprising:
(a) one or more reaction tubes comprising Reaction Reagent;
(b) one or more master tubes comprising Reaction Medium;
(c) a readout reagent comprising a reporter entity and a reporter buffer; and
(d) an instruction for use of the kit.

77. The kit of claim 76, further comprising one or more evaluation strips and a plate adapter.

78. The kit of claim 76 or 77, further comprising a blank sample.


SEQUENCE LISTING (4 sequences)

SEQ ID NO: 1 - protein, 1640 aa, synthetic construct
  REGION 1..1640: note = Strain Annexin
MRQVWFSWIVGLFLCFFNVSSAAPVNTTTEDETAQIPAEAVIGYLDLEGDFDVAVLPFSNSTNNGLLFINTTIASIAAKEEGVSLDKREAEAGSGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDDGSGSGSGGGGSGGGGSGGGGSGGGGSGGGGSGSGGGSGGGGSGGSGSDYKDHDGDYKDHDIDYKDDDYKDYKDHDGDYKDHDIDYKDDDYKGSGTTKSSTTTSSTSESSTSSSTTAPATPTTTSCTKEKPTPPTTTSCTKEKPTPPHHDTTPCTKKKTTTSKTCTKKTTTPVPTPSSSTTESSSAPVPTPSSSTTESSSAPVTSSTTESSSAPVPTPSSSTTESSSAPVTSSTTESSSAPVTSSTTESSSAPVPTPSSSTTESSSAPVTSSTTESSSAPVTSSTTESSSAPVTSSTTESSSAPVTSSTTESSSAPVPTPSSSTTESSSAPVTSSTTESSSAPVPTPSSSTTESSSAPVTSSTTESSSAPVPTPSSSTTESSSAPAPTPSSSTTESSSAPVTSSTTESSSAPVPTPSSSTTESSSTPVTSSTTESSSAPVPTPSSSTTESSSAPVPTPSSSTTESSSAPAPTPSSSTTESSSAPVTSSTTESSSAPVPTPSSSTTESSSAPVPTPSSSTTESSSAPVPTPSSSTTESSSAPVTSSTTESSSAPVTSSTTESSSAPVPTPSSSTTESSSAPVPTPSSSTTESSSAPVPTPSSSTTESSSAPVTSSTTESSSAPVPTPSSSTTESSSAPVPTPSSSTTESSSAPVPTPSSSTTESSVAPVPTPSSSSNITSSAPSSTPFSSSTESSSVPVPTPSSSTTESSSAPVSSSTTESSVAPVPTPSSSSNITSSAPSSIPFSSTTESFSTGTTVTPSSSKYPGSQTETSVSSTTETTIVPTKTTTSVTTPSTTTITTTVCSTGTNSAGETTSGCSPKTVTTTVPTTTTTSVTTSSTTTITTTVCSTGTNSAGETTSGCSPKTITTTVPCSTSPSETASESTTTSPTTPVTTVVSTTVVTTEYSTSTKPGGEITTTFVTKNIPTTYLTTIAPTPSVTTVTNFTPTTITTTVCSTGTNSAGETTSGCSPKTVTTTVPCSTGTGEYTTEATTLVTTAVTTTVVTTESSTGTNSAGKTTTGYTTKSVPTTYVTTLAPSAPVTPATNAVPTTITTTECSAATNAAGETTSVCSAKTIVSSASAGENTAPSATTPVTTAIPTTVITTESSVGTNSAGETTTGYTTKSIPTTYITTLIPGSNGAKNYETVATATNPISIKTTSQLATTASASSVAPVVTSPSLTGPLQSASGSAVATYSVPSISSTYQGAANIKVLGNFMWLLLALPVVF

SEQ ID NO: 2 - protein, 1398 aa, synthetic construct
  REGION 1..1398: note = Strain EGFR
MRQVWFSWIVGLFLCFFNVSSAAPVNTTTEDETAQIPAEAVIGYLDLEGDFDVAVLPFSNSTNNGLLFINTTIASIAAKEEGVSLDKREAEAGSDYKDHDGDYKDHDIDYKDDDDKGSGGAEAAAKEAAAKEAAAKAGSAGSDYKDHDGDYKDHDIDYKDDDDKGSGGAEAAAKEAAAKEAAAKAGSAGSDYKDHDGDYKDHDIDYKDDDDKGSGGAEAAAKEAAAKEAAAKAGSAGSGSGGAEAAAKEAAAKEAAAKAGSAAGGSGGTKSSTTTSSTSESSTSSSTTAPATPTTTSCTKEKPTPPTTTSCTKEKPTPPHHDTTPCTKKKTTTSKTCTKKTTTPVPTPSSSTTESSSAPVPTPSSSTTESSSAPVTSSTTESSSAPVPTPSSSTTESSSAPVTSSTTESSSAPVTSSTTESSSAPVPTPSSSTTESSSAPVTSSTTESSSAPVTSSTTESSSAPVTSSTTESSSAPVTSSTTESSSAPVPTPSSSTTESSSAPVTSSTTESSSAPVPTPSSSTTESSSAPVTSSTTESSSAPVPTPSSSTTESSSAPAPTPSSSTTESSSAPVTSSTTESSSAPVPTPSSSTTESSSTPVTSSTTESSSAPVPTPSSSTTESSSAPVPTPSSSTTESSSAPAPTPSSSTTESSSAPVTSSTTESSSAPVPTPSSSTTESSSAPVPTPSSSTTESSSAPVPTPSSSTTESSSAPVTSSTTESSSAPVTSSTTESSSAPVPTPSSSTTESSSAPVPTPSSSTTESSSAPVPTPSSSTTESSSAPVTSSTTESSSAPVPTPSSSTTESSSAPVPTPSSSTTESSSAPVPTPSSSTTESSVAPVPTPSSSSNITSSAPSSTPFSSSTESSSVPVPTPSSSTTESSSAPVSSSTTESSVAPVPTPSSSSNITSSAPSSIPFSSTTESFSTGTTVTPSSSKYPGSQTETSVSSTTETTIVPTKTTTSVTTPSTTTITTTVCSTGTNSAGETTSGCSPKTVTTTVPTTTTTSVTTSSTTTITTTVCSTGTNSAGETTSGCSPKTITTTVPCSTSPSETASESTTTSPTTPVTTVVSTTVVTTEYSTSTKPGGEITTTFVTKNIPTTYLTTIAPTPSVTTVTNFTPTTITTTVCSTGTNSAGETTSGCSPKTVTTTVPCSTGTGEYTTEATTLVTTAVTTTVVTTESSTGTNSAGKTTTGYTTKSVPTTYVTTLAPSAPVTPATNAVPTTITTTECSAATNAAGETTSVCSAKTIVSSASAGENTAPSATTPVTTAIPTTVITTESSVGTNSAGETTTGYTTKSIPTTYITTLIPGSNGAKNYETVATATNPISIKTTSQLATTASASSVAPVVTSPSLTGPLQSASGSAVATYSVPSISSTYQGAANIKVLGNFMWLLLALPVVF

SEQ ID NO: 3 - protein, 1741 aa, synthetic construct
  REGION 1..1741: note = Strain Bar1
MSAINHLCLKLILASFAIINTITALTNDGTGHLEFLLQHEEEMYYATTLDIGTPSQSLTVLFDTGSADFWVMDSSNPFCLPNSNTSSYSNATYNGEEVKPSIDCRSMSTYNEHRSSTYQYLENGRFYITYADGTFADGSWGTETVSINGIDIPNIQFGVAKYATTPVSGVLGIGFPRRESVKGYEGAPNEYYPNFPQILKSEKIIDVVAYSLFLNSPDSGTGSIVFGAIDESKFSGDLFTFPMVNEYPTIVDAPATLAMTIQGLGAQNKSSCEHETFTTTKYPVLLDSGTSLLNAPKVIADKMASFVNASYSEEEGIYILDCPVSVGDVEYNFDFGDLQISVPLSSLILSPETEGSYCGFAVQPTNDSMVLGDVFLSSAYVVFDLDNYKISLAQANWNASEVSKKLVNIQTDGSISGAKIATAEPWSTNEPFTVTSDIYSSTGCKSRPFLQSSTASSLIAETNVQSRNCSTKMPGTRSTTVLSKPTQNSAMHQSTGAVTQTSNETKLELSSTMANSGSVSLPTSNSIDKEFEHSKSQTTSDPSVAEHSTFNQTFVHETKYRPTHKTVITETVTKYSTVLINVCKPTYGSGGTKSSTTTSSTSESSTTTSSTSESSTTTSSTSESSTSSSTTAPATPTTTSCTKEKPTPPTTTSCTKEKPTPPHHDTTPCTKKKTTTSKTCTKKTTTPVPTPSSSTTESSSAPVPTPSSSTTESSSAPVTSSTTESSSAPVPTPSSSTTESSSAPVTSSTTESSSAPVTSSTTESSSAPVPTPSSSTTESSSAPVTSSTTESSSAPVTSSTTESSSAPVTSSTTESSSAPVTSSTTESSSAPVPTPSSSTTESSSAPVTSSTTESSSAPVPTPSSSTTESSSAPVTSSTTESSSAPVPTPSSSTTESSSAPAPTPSSSTTESSSAPVTSSTTESSSAPVPTPSSSTTESSSTPVTSSTTESSSAPVPTPSSSTTESSSAPVPTPSSSTTESSSAPAPTPSSSTTESSSAPVTSSTTESSSAPVPTPSSSTTESSSAPVPTPSSSTTESSSAPVPTPSSSTTESSSAPVTSSTTESSSAPVTSSTTESSSAPVPTPSSSTTESSSAPVPTPSSSTTESSSAPVPTPSSSTTESSSAPVTSSTTESSSAPVPTPSSSTTESSSAPVPTPSSSTTESSSAPVPTPSSSTTESSVAPVPTPSSSSNITSSAPSSTPFSSSTESSSVPVPTPSSSTTESSSAPVSSSTTESSVAPVPTPSSSSNITSSAPSSIPFSSTTESFSTGTTVTPSSSKYPGSQTETSVSSTTETTIVPTKTTTSVTTPSTTTITTTVCSTGTNSAGETTSGCSPKTVTTTVPTTTTTSVTTSSTTTITTTVCSTGTNSAGETTSGCSPKTITTTVPCSTSPSETASESTTTSPTTPVTTVVSTTVVTTEYSTSTKPGGEITTTFVTKNIPTTYLTTIAPTPSVTTVTNFTPTTITTTVCSTGTNSAGETTSGCSPKTVTTTVPCSTGTGEYTTEATTLVTTAVTTTVVTTESSTGTNSAGKTTTGYTTKSVPTTYVTTLAPSAPVTPATNAVPTTITTTECSAATNAAGETTSVCSAKTIVSSASAGENTAPSATTPVTTAIPTTVITTESSVGTNSAGETTTGYTTKSIPTTYITTLIPGSNGAKNYETVATATNPISIKTTSQLATTASASSVAPVVTSPSLTGPLQSASGSAVATYSVPSISSTYQGAANIKVLGNFMWLLLALPVVF

SEQ ID NO: 4 - protein, 472 aa, synthetic construct
  REGION 1..472: note = Modified GPA1
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKDSGIL